SEQUENCE LISTING (29 sequences)

SEQ ID NO: 1 - DNA, 647 bp, Glycine max
aagatggtgcagcctgcttcctaaattttggtacattatcaagtggatgtgatggtgcccctctgcttcctctgctgtttattattgtaaacataggtttcaatattgcattgcttcatctcctcaagatctcttcagctgttgtatcttgtcttgcttccacattttcagtcccaatatccatctacgtgttcaccatgccattgccataccttggtgttgcctcctctcttccaacaggctttatggcaggggccattatcctcattttgggcttactcatttatgcttggaccccttcaaatggttcctcgggtgcttccttctcaacttcctccacctagagaggctagaatgagttgacatgtcattgcagatagtacaacaccacaaggaactaattcaggttcgcttttaggagacggctataagaaggagaaagaaatagggcgttcttgtaagttgtaatagttgttcgtaagcattttttatgagctaagcttaagtaagaaagagactagactatagatagaacaggttccaagttcaatttttatgtaagctaaggaaagtaaatagagaataaaagtcactttgttgacagaggaaatgatattggaccatttggatgcaaaaaaaaaaaaaaa

SEQ ID NO: 2 - DNA, 477 bp, Glycine max
gtgcagcctgcttcctaaattttggtacattatcaagtggatgtgatggtgcccctctgcttcctctgctgtttattattgtaaacataggtttcaatattgcattgcttcatctcctcaagatctcttcagctgttgtatcttgtcttgcttccacattttcagtcccaatatccatctacgtgttcaccatgccattgccataccttggtgttgcctcctctcttccaacaggctttatggcaggggccattatcctcattttgggcttactcatttatgcttggaccccttcaaatggttcctcgggtgcttccttctcaacttcctccacctagagaggctagaatgagttgacatgtcattgcagatagtacaacaccacaaggaactaattcaggttcgcttttaggagacggctataagaaggagaaagaaatagggcgttcttgtaagttgtaatagttgttcgtaagc

SEQ ID NO: 3 - protein, 113 aa, Glycine max
DGAACFLNFGTLSSGCDGAPLLPLLFIIVNIGFNIALLHLLKISSAVVSCLASTFSVPISIYVFTMPLPYLGVASSLPTGFMAGAIILILGLLIYAWTPSNGSSGASFSTSST

SEQ ID NO: 4 - DNA, 29 bp, Artificial
  primer sequence
ttaattaagacgtcgtgcagcctgcttcc

SEQ ID NO: 5 - DNA, 33 bp, Artificial
  primer sequence
ctcgagggcgcgccgcttacgaacaactattac

SEQ ID NO: 6 - DNA, 1449 bp, Arabidopsis thaliana
cataacggagtatgatattaaatgaacactttctgttcgtatcaaatggtatcgaatattattagagtgatcattcagaagaaaaaaagagagagaagaaaacctacagtgtaaacatttttttttttgctaaatacctacagtgtaaacatgaagtgctataatttctgcaaatagaaatcaagaacagaaagagttgcttggaggaaaagaaatagaaaattaagaaatctagtgatgtaataaatctttccataaaatcaaatgtttggtccaaagtattagttaaataattaggccactattcttgacaactctttttaacaaactcttctatattttctcgtggtacatatgctgaaaaagatgtatgtctaatccataatatatctgtataatgcgactttcattatctattagtacgacttctaacctagaagataacaagcattagctagggcatcaaaatcaacgtggaaaaacctacgaaaagcacgaagtgattaatctgtgtaggggtggcgtaagggtaaagactaaagactgagaatctagggttcaaggcgtaaacttgttctgctttttgggtttcattttattggcgaacaacattgatgtgtgtggaccatttggtgttcagggattgagacaagataatatgtttgctctcaccttctaggattactcgggtgctaagactcacttagtactattgctatatcgatatactagttcattaccaaaaaatggagtcttcaaatttcgagttccaatatctgaaagcattgtttaaagagatttgttttctccctgcacaattagtttataacttcatatatacacaatcttatcaatttacaaccaggtgtgtgtgaaccttcacataatctctcttattcattcatgtatatatccaataaaagttcgatatgtgaaattatatatctccatctaatgttagactattcccgggtcttgactataaatttaaagtattagacgagctaattatatttagcacaaacaatttcttctgtaacagtgtcacgcttatcactaccaaagaataaacactgatctgttttaatctcttattttctcacccatattcaaagtcaactattgcaagacttcgagataattaatttgatggctatactatttacttgacatttgggaaaatatattttcgctgataaatttggtttttacttctctctccgacggatatagaaacaattcaattacatgcgaaaatgataattcaaccctataaaccaaaacaaataacagaatgcacatttttttcaacgcgttaggtcacctatctttcactttagaacatcccttcacgtctctatataaacctcgactctgttatcctttgttcttcaagtacaacaatcaactctaagtctattatattcaagtctttgttttaacctaaca

SEQ ID NO: 7 - DNA, 1352 bp, Glycine max
gatgttcttctattgattcctcaatttctccaactcaaaactaagtttcgtctttgtgatctcttgattatattatatcgatttgtgaattgtttttccacaaaaaaaaaattcattcaaaccatggcttcattatgccggcggttatccgccgccgcaccggccggtcacttccgattccatcggcccgccgggatttgcgcggttcgatacggttattccgtccggcggccaatgcggttggttgcggcggggaccaccgtgggcggcggcgcgtgggtgacatccgatgacgtcggagggagggaggagaaggtggggccctgctcctacgccgtggaggatcggagggtggcggaagatggcggaagcgatgcggtggtaggaagtaggaaccgggtggtggaagtggcggcggcggcggttgcaacggtggtgctgggtgttgggaaccgggttctgtataagttggctttggttccgttgaagcactaccctttcttccttgctcaacttgccactttcggatatgtaatagtgtactttgcgattttgtatattcgacaccatgcgggcattgttactgatgagatgttagatgctccaaaggctccatttatagttgttggtctattggaggctctcgctgctgccactggaatggccgcaggagcaattctctctggagcttcgattccaattttatctcagacttttctagtgtggcaaatactcctgtcaattatttttcttgggagaagatataaagtcaaccaattacttggatgctttcttgtaaccattggtgtagttgttactgtagcaagtggagctggtgctgggaatttattaaaggaaggtggtatgttttggagtcttttgatgatagtttcatttttcctccaagcggctgataccgtgctgaaggaaattatctttttggattcatcccgaaaattgaagggaggttgttgtatggacctttttgttgtcaattcgtacggatctgctttccaagcactattcgtgtgccttcttctccccttcttgtcaaaattatggggcattcccttcagtcaactaccaaactaccttaaagatggtgcagcctgcttcctaaattttggtacattatcaagtggatgtgatggtgcccctctgcttcctctgctgtttattattgtaaacataggtttcaatattgcattgcttcatctcctcaagatctcttcagctgttgtatcttgtcttgcttccacattttcagtcccaatatccatctacgtgttcaccatgccattgccataccttggtgttgcctcctctcttccaacaggctttatggcaggggc

SEQ ID NO: 8 - DNA, 1743 bp, Glycine max
gatgttcttctattgattcctcaatttctccaactcaaaactaagtttcgtctttgtgatctcttgattatattatatcgatttgtgaattgtttttccacaaaaaaaaaattcattcaaaccatggcttcattatgccggcggttatccgccgccgcaccggccggtcacttccgattccatcggcccgccgggatttgcgcggttcgatacggttattccgtccggcggccaatgcggttggttgcggcggggaccaccgtgggcggcggcgcgtgggtgacatccgatgacgtcggagggagggaggagaaggtggggccctgctcctacgccgtggaggatcggagggtggcggaagatggcggaagcgatgcggtggtaggaagtaggaaccgggtggtggaagtggcggcggcggcggttgcaacggtggtgctgggtgttgggaaccgggttctgtataagttggctttggttccgttgaagcactaccctttcttccttgctcaacttgccactttcggatatgtaatagtgtactttgcgattttgtatattcgacaccatgcgggcattgttactgatgagatgttagatgctccaaaggctccatttatagttgttggtctattggaggctctcgctgctgccactggaatggccgcaggagcaattctctctggagcttcgattccaattttatctcagacttttctagtgtggcaaatactcctgtcaattatttttcttgggagaagatataaagtcaaccaattacttggatgctttcttgtaaccattggtgtagttgttactgtagcaagtggagctggtgctgggaatttattaaaggaaggtggtatgttttggagtcttttgatgatagtttcatttttcctccaagcggctgataccgtgctgaaggaaattatctttttggattcatcccgaaaattgaagggaggttgttgtatggacctttttgttgtcaattcgtacggatctgctttccaagcactattcgtgtgccttcttctccccttcttgtcaaaattatggggcattcccttcagtcaactaccaaactaccttaaagatggtgcagcctgcttcctaaattttggtacattatcaagtggatgtgatggtgcccctctgcttcctctgctgtttattattgtaaacataggtttcaatattgcattgcttcatctcctcaagatctcttcagctgttgtatcttgtcttgcttccacattttcagtcccaatatccatctacgtgttcaccatgccattgccataccttggtgttgcctcctctcttccaacaggctttatggcaggggccattatcctcattttgggcttactcatttatgcttggaccccttcaaatggttcctcgggtgcttccttctcaacttcctccacctagagaggctagaatgagttgacatgtcattgcagatagtacaacaccacaaggaactaattcaggttcgcttttaggagacggctataagaaggagaaagaaatagggcgttcttgtaagttgtaatagttgttcgtaagcattttttatgagctaagcttaagtaagaaagagactagactatagatagaacaggttccaagttcaatttttatgtaagctaaggaaagtaaatagagaataaaagtcactttgttgacagaggaaatgatattggaccatttggatgcaaaaaaaaaaaaaaa

SEQ ID NO: 9 - DNA, 1317 bp, Glycine max
atggcttcattatgccggcggttatccgccgccgcaccggccggtcacttccgattccatcggcccgccgggatttgcgcggttcgatacggttattccgtccggcggccaatgcggttggttgcggcggggaccaccgtgggcggcggcgcgtgggtgacatccgatgacgtcggagggagggaggagaaggtggggccctgctcctacgccgtggaggatcggagggtggcggaagatggcggaagcgatgcggtggtaggaagtaggaaccgggtggtggaagtggcggcggcggcggttgcaacggtggtgctgggtgttgggaaccgggttctgtataagttggctttggttccgttgaagcactaccctttcttccttgctcaacttgccactttcggatatgtaatagtgtactttgcgattttgtatattcgacaccatgcgggcattgttactgatgagatgttagatgctccaaaggctccatttatagttgttggtctattggaggctctcgctgctgccactggaatggccgcaggagcaattctctctggagcttcgattccaattttatctcagacttttctagtgtggcaaatactcctgtcaattatttttcttgggagaagatataaagtcaaccaattacttggatgctttcttgtaaccattggtgtagttgttactgtagcaagtggagctggtgctgggaatttattaaaggaaggtggtatgttttggagtcttttgatgatagtttcatttttcctccaagcggctgataccgtgctgaaggaaattatctttttggattcatcccgaaaattgaagggaggttgttgtatggacctttttgttgtcaattcgtacggatctgctttccaagcactattcgtgtgccttcttctccccttcttgtcaaaattatggggcattcccttcagtcaactaccaaactaccttaaagatggtgcagcctgcttcctaaattttggtacattatcaagtggatgtgatggtgcccctctgcttcctctgctgtttattattgtaaacataggtttcaatattgcattgcttcatctcctcaagatctcttcagctgttgtatcttgtcttgcttccacattttcagtcccaatatccatctacgtgttcaccatgccattgccataccttggtgttgcctcctctcttccaacaggctttatggcaggggccattatcctcattttgggcttactcatttatgcttggaccccttcaaatggttcctcgggtgcttccttctcaacttcctccacctag

SEQ ID NO: 10 - protein, 438 aa, Glycine max
MASLCRRLSAAAPAGHFRFHRPAGICAVRYGYSVRRPMRLVAAGTTVGGGAWVTSDDVGGREEKVGPCSYAVEDRRVAEDGGSDAVVGSRNRVVEVAAAAVATVVLGVGNRVLYKLALVPLKHYPFFLAQLATFGYVIVYFAILYIRHHAGIVTDEMLDAPKAPFIVVGLLEALAAATGMAAGAILSGASIPILSQTFLVWQILLSIIFLGRRYKVNQLLGCFLVTIGVVVTVASGAGAGNLLKEGGMFWSLLMIVSFFLQAADTVLKEIIFLDSSRKLKGGCCMDLFVVNSYGSAFQALFVCLLLPFLSKLWGIPFSQLPNYLKDGAACFLNFGTLSSGCDGAPLLPLLFIIVNIGFNIALLHLLKISSAVVSCLASTFSVPISIYVFTMPLPYLGVASSLPTGFMAGAIILILGLLIYAWTPSNGSSGASFSTSST

SEQ ID NO: 11 - RNA, 44 nt, Artificial
  RNA oligonucleotide
cgacuggagcacgaggacacugacauggacugaaggaguagaaa

SEQ ID NO: 12 - DNA, 23 bp, Artificial
  primer sequence
cgactggagcacgaggacactga

SEQ ID NO: 13 - DNA, 26 bp, Artificial
  primer sequence
ggacactgacatggactgaaggagta

SEQ ID NO: 14 - DNA, 28 bp, Artificial
  primer sequence
ccgaggaaccatttgaaggggtccaagc

SEQ ID NO: 15 - DNA, 28 bp, Artificial
  primer sequence
gcccctgccataaagcctgttggaagag

SEQ ID NO: 16 - protein, 453 aa, Vitis vinifera
MSASCRRLTAGVRVPMVARQVLGASEISRLCGVRMNRRKGIVLRGRRLVVAEAEAMGRGGVRVRSDGGGEERVEKWSYGSEDRRRGDLVVVEEKEDVGCAGGGCEGDRRMKVVIAAAFTVVLGVGNRVLYKLALVPLKHYPFFLAQLATVGYVLVYFSILSLRYNAGIVTDEMLSLPKTPYVAVGLLEALGAATGMAAGAILSGASIPILSQSFLVWQLLLSAIFLGRRYKVNQLLGCFLVAIGVIITVASGSSAGASLKGAGIFWSLLMMVSFLFQAADTVLKERIFLKAAERLKGGSVDLFVVNSYGSAFQALFICLLLPFLSKLWGVPFSHLPNYLKDGAACFLNIGSLSSGCDGAPLLPLLFVVVNMGFNISLLHLLKISSAVVSCLASTFSVPIAVYMFTLPLPYLGVASSLPPAFVTGAIILLVGLMIYAWTPPSMDLNSSSSPSIH

SEQ ID NO: 17 - DNA, 1362 bp, Vitis vinifera
atgtctgcctcttgccgccggctaaccgccggcgttcgtgttccaatggttgccaggcaggttctgggagccagcgagatttcgaggctttgtggagttcgaatgaaccggcggaaggggatcgttttgagggggaggcggctggtggtggcggaggcggaggctatgggaagaggaggagtgcgggtgagatcggatgggggaggtgaggagagggtggagaagtggtcgtacgggtcggaggatcggcggaggggcgatttggtggtggtggaggaaaaggaggatgttggctgcgctggtggcggttgtgagggtgatcggaggatgaaagtggttatcgcagcggcgtttacggtggtgctgggcgtagggaatcgagtgctgtacaagctggccttggttcctctcaaacattacccattcttcctcgctcagcttgccactgtcggatatgtacttgtgtacttctctatcttgtctctgcgttacaatgctggaattgttacagatgagatgctttccttgccaaagactccctatgttgctgtgggccttttagaggctcttggtgctgctactggaatggcagctggagcaattctttctggagcatcaattccaattttatctcagagctttcttgtctggcaacttctcctatcagctatttttcttggtaggagatacaaagtcaaccaattacttggatgttttcttgtggctattggtgtaataataactgtagcaagtggatctagtgctggtgcttcattgaagggagctggcatattttggagtcttttgatgatggtttcctttttgtttcaagcggctgatacagtcttgaaggagagaattttcttgaaggctgctgagcgattaaagggaggttcagtggatctatttgttgtaaattcctatggatctgctttccaagcattgttcatatgccttctcctgcccttcttatcaaagttatggggtgttccatttagtcatctgccaaactaccttaaagatggtgcagcctgctttctgaacatcggttctttgtccagtggatgtgatggtgcaccgctactgccattgctctttgttgtagtcaacatgggcttcaacatatcactgctgcatctcctcaagatatcttctgctgtggtatcttgccttgcatccactttttcagtgccaattgctgtgtacatgttcacactgccgcttccatatcttggtgtcgcatcttctcttccaccagcttttgtgacaggtgctattatccttctggtgggcctaatgatttatgcatggacaccgccgtcaatggatctgaacagttcttcctcaccttcaatccactag

SEQ ID NO: 18 - protein, 422 aa, Vitis vinifera
MAFSCAVSCVRFHVIPPKPLTLHTYTAHLSPFSPLISMHISQNPRYHLHFPSRSPLHGVSSHNSPKTPNFRVRASADNSQTSSSNTGLVIVCSAITVILAVVNRVFYKLALVPLKQYPFFLAQFTTFGYAAIYFSILYIRYRAGIVTDEMIALPKSRFMAIGILEALGVASGMASAAMLPGPAIPLLNQTFLVWQLALSTLILGRKYSFNQILGCFLVAAGVVTAVASGSNGDQMLSGIEFIWPALMIASSAFQAGASIIKEFVFVDAATRLKGKLLDIFVVNSFGSGFQALFVLLLLPLLSNFRGIPFPQLPSYLKAGAGCFLNIGSNIPGCDGAPLLPLLYLATNIAFNISLLNLVKISSAVVSTLAAMASVPISIYVLSLPLPYLPQGASLSPFFLFGGVILLLGLLLYNIPQPAKQAP

SEQ ID NO: 19 - DNA, 1269 bp, Vitis vinifera
atggccttctcgtgcgcggtgtcctgcgtccgtttccatgtcattcctcctaaacccttaactcttcatacctacaccgcccatctctcacccttctctcctctaatttcaatgcatatttctcaaaaccctagataccatcttcattttccttccagatcacccctccatggagtttcaagtcacaattcaccgaaaacccccaacttcagagtccgcgcatccgccgacaattcccaaacttcatcctccaacaccggactagtaatcgtctgctcggcaatcactgtaattttggcggttgtcaatcgcgtgttttacaagcttgctcttgttcctctgaagcaatacccattctttttagcccagttcactactttcgggtatgcggctatttatttttctatactatatatacggtatcgtgcggggattgtaacggatgaaatgatagcccttccaaaatcacgttttatggcaatcggtattctagaagcccttggtgttgcttctgggatggcttctgcagctatgcttcctggaccagctatacccttattgaatcagacatttttggtttggcagctggctctttctactcttattttggggagaaaatactccttcaatcaaattcttggatgctttcttgtagctgctggtgtagtaaccgctgttgcgagtgggtcaaatggtgatcaaatgctgtcaggaattgagtttatatggccagcattaatgatagcttcaagtgcattccaagctggtgcatctattatcaaggaatttgtttttgttgatgctgcaacccggcttaaggggaagcttcttgacatatttgttgtcaattcttttggatccggattccaggctctttttgtgcttctccttctgcctttgctgtcaaacttcagaggtataccttttccacagctcccttcatacctaaaagctggtgctggttgctttttgaatattggatccaatataccaggttgtgatggagctccattgctgcccctgctttatttggctaccaatatagctttcaacatatcattgcttaatctagtaaaaatttcctctgcagtagtttctactcttgctgcaatggcatcagtgccaatttcaatttatgttcttagccttccattgccatatctcccacaaggtgccagcttgagtcccttttttctctttggtggtgtgattctcctgttgggccttcttctatacaacatacctcagcctgccaagcaagccccttga

SEQ ID NO: 20 - protein, 343 aa, Oryza sativa
MSSSPAIAAASAAVVALAVANRVLYKLALVPLKQYPFFLAQLTTFGYVAVYFSILYARYRAGVVTGDMLALPKRRLAAIGLLEALGLAAGMSAGAMLPGPAIPILSQSFLVWQLIFSALLLGRTYSMRQIIGCFLVASGVILAVASGANEGQFLSEVKFIWLALMVASSAFQAGASILKESVFIDGAKRLKGRRPDIFVVNSFGSGFQALFVFLLLPLLSNLKGIKFAELPAYLNGGAECFLNVDDSLIDCGGAPFLPLLFILVNMAFNIALLNLVKLSSALVASLTATSAVPISIYILSLPLPYIPHGAELSSSFILGGVVLLMGLIIYNLPQSSKKQSKIE

SEQ ID NO: 21 - DNA, 1032 bp, Oryza sativa
atgagctcgtcgccggcgatcgccgcggcgtcggcggcagtggtggcgctggccgtcgccaaccgcgtgctgtacaagctcgcgctcgtgccgctcaagcagtacccgttcttcctcgcccagctcaccaccttcgggtacgtcgccgtgtacttctcgatactctacgcgaggtaccgcgcgggggtggtgacgggggacatgctggcgctgccgaagcgccggttggccgccatcggcttgctggaggccctcgggcttgcggccggcatgtctgcaggagctatgcttcctgggcctgctattcctatactgtcccagtcattcctggtgtggcagcttatcttctctgcgctgctgttgggaagaacgtattcaatgaggcaaatcattggttgtttccttgttgcttctggtgtgattcttgctgttgcaagtggagcgaatgagggtcaatttctgtctgaagtcaagtttatttggctagcactgatggttgcatcatcagcatttcaagcaggtgcttcaattctgaaggaatctgttttcattgatggtgcaaagcgtcttaaggggaggcgcccagatatctttgtggttaattcgtttgggtcaggatttcaggctctctttgtctttcttcttctcccacttctttctaatttgaagggaataaagtttgctgagcttcctgcttatttaaatggtggtgctgagtgcttcctaaatgtcgatgatagtctgattgattgtggaggagctccatttctaccattgctgtttatattggtgaatatggccttcaatatcgcattgctcaatttggtgaagctgtcatctgcgctggttgcttcgcttactgctacttcagcagtgccaatatcaatctacatactttcacttccgttgccctacatccctcatggcgcagagttaagttcatctttcatcctaggcggtgtggtattgctgatggggctgattatatataaccttccgcaatcatcgaagaaacaatccaagattgagtga

SEQ ID NO: 22 - protein, 495 aa, Oryza sativa
MELLPTVRRREAVRAAHVRRIEAAAWLGARRATRREDAAARCAAAGEVVGSAAGVGRSAGMEVAIATAAVVAMGTGNRVLYKLALVPLRDYPFFLAQLATFGLCDGCLVLGVHLSGRITGRDFLLVNISIPVRLCAFFLVGGRARELAFQASWRLYQQHQGWLLETYLVWQLLLSAIFLKRRYRINEITGCFLVTVGVIITVASGSSAGASLKGTGILWPLLMIISFFLQAADTVLKEIIFLNAAKKLKGGSVDLFVVNSYGSAYQALFMCLLLPFLSKLWGVPFHQLPTYIRDGTACFLNMGSLSSGCEGAPLLPLLFVLVNMGFNISLLHLLKISSAVVSSLASTFSVPLSIYAFTLPLPYIGVASTLPPGFVAGAMFLDGTRTREKRRSIITTGMKSKNGGGGGSGGNTNGSHRRITAAAAINIIRTLLSILASPAAVDWTASSGRRLTGCSTLRCSSVDLWVPSLGRRGFRWSPGVRQGGHDPDKHRVMGI

SEQ ID NO: 23 - DNA, 1488 bp, Oryza sativa
atggagctcctccctaccgtgaggcggagggaggcggtgcgggcggcgcacgtgcggcggatcgaggcggcggcgtggttgggggcgaggagggcgacccggagggaggatgcggcggcgaggtgcgcggcggccggggaggtggtggggagcgccgctggggtcgggaggagcgccgggatggaggtggcaatcgccaccgcggcggtcgtggccatgggtacggggaaccgcgtcctctacaagctcgccctcgtgccgctccgcgactaccccttcttcctcgcccagctcgccacgttcggcctttgcgatgggtgcctggtgttgggggtccacttgtctgggagaataactggacgagattttttgcttgttaatatctcaattcctgtacggctatgcgctttttttctggtgggtggtcgagctcgtgagctggccttccaggcctcttggaggctttatcagcagcatcagggatggctgctggagacgtatcttgtttggcagcttcttttatctgctatttttttgaagaggcgctatagaataaatgagataactggatgctttcttgtgacggttggtgtaataataactgtagcaagtggatctagtgctggtgcttcattaaaaggtactggaattttgtggccactgctcatgataatatcgttctttctccaagccgctgatacagtattgaaggagataatatttctgaatgctgccaagaaattgaagggtggctcggttgatctttttgtcgtcaactcatatggctctgcttatcaagctcttttcatgtgtctcttgttgccattcttgtcaaagttatggggagtgccattccatcaactaccaacatacatcagagatggcaccgcctgctttctgaacatgggatcactatcttctggctgtgaaggggcaccactattaccactactatttgtgttggttaatatgggcttcaatatatcacttctacacttactaaagatttcttcagcagttgtatcttctctggcctctacattttcagttccactgtccatctacgccttcaccctaccgttgccgtacatcggtgtggcgtctactctccctcctggctttgttgcaggcgccatgtttctagacgggacaagaaccagggagaaaagaaggagcatcatcacgacggggatgaagagcaagaatggcggcggcggcggtagcggcggcaacaccaatgggagccacaggaggatcaccgccgccgccgccatcaacatcatcaggacgctcttgtccatcttggcctcccctgcagctgttgactggactgcctcctccggccggaggctcaccggttgctccactctccggtgttcatccgtcgacttgtgggtcccttctctgggacgtcgaggattcaggtggtccccgggggtgcggcagggaggccatgaccccgacaagcatcgcgtgatggggatctag

SEQ ID NO: 24 - protein, 531 aa, Oryza sativa
MASSTTTAPPVSCRPSTARGRLLRLLPANGPAAAMELLPTVRRREAVRAAHVRRIEAAAWLGARRATRREDAAARCAAAGEVVGSAAGVGRSAGMEVAIATAAVVAMGTGNRVLYKLALVPLRDYPFFLAQLATFGYVVVYFSILYLRHQAGIVTDEMLSLPQKPFLAVGLLEALSAASGMAAGAVLSGASIPILSQTYLVWQLLLSAIFLKRRYRINEITGCFLVTVGVIITVASGSSAGASLKGTGILWPLLMIISFFLQAADTVLKEIIFLNAAKKLKGGSVDLFVVNSYGSAYQALFMCLLLPFLSKLWGVPFHQLPTYIRDGTACFLNMGSLSSGCEGAPLLPLLFVLVNMGFNISLLHLLKISSAVVSSLASTFSVPLSIYAFTLPLPYIGVASTLPPGFVAGAVLGDASTVSRRDKNQGEKKEHHHDGDEEQEMAAAAVAAATPIGEPQGGSPPPPPSTSSGRSCPSWPPLQLLTGLPPPAGGSPVAPLSGVHPSTCGSLLWEVEDSGGPRGCGREAMTPTSIG

SEQ ID NO: 25 - DNA, 1596 bp, Oryza sativa
atggcgtcgtcgacgacgacggcgccccccgtctcgtgccgcccctccacggcgcgggggcggctgctccgcctcctccccgcgaacgggccggcggctgcgatggagctcctccctaccgtgaggcggagggaggcggtgcgggcggcgcacgtgcggcggatcgaggcggcggcgtggttgggggcgaggagggcgacccggagggaggatgcggcggcgaggtgcgcggcggccggggaggtggtggggagcgccgctggggtcgggaggagcgccgggatggaggtggcaatcgccaccgcggcggtcgtggccatgggtacggggaaccgcgtcctctacaagctcgccctcgtgccgctccgcgactaccccttcttcctcgcccagctcgccacgttcggctatgtggttgtctatttctcgatcctgtatcttcgccatcaagctggcattgtcactgatgagatgctgtctctaccacaaaaaccttttctggctgtaggcctcttggaggctttatcagcagcatcagggatggctgctggagccgttctttctggggcttcgataccaatactgtcacagacgtatcttgtttggcagcttcttttatctgctatttttttgaagaggcgctatagaataaatgagataactggatgctttcttgtgacggttggtgtaataataactgtagcaagtggatctagtgctggtgcttcattaaaaggtactggaattttgtggccactgctcatgataatatcgttctttctccaagccgctgatacagtattgaaggagataatatttctgaatgctgccaagaaattgaagggtggctcggttgatctttttgtcgtcaactcatatggctctgcttatcaagctcttttcatgtgtctcttgttgccattcttgtcaaagttatggggagtgccattccatcaactaccaacatacatcagagatggcaccgcctgctttctgaacatgggatcactatcttctggctgtgaaggggcaccactattaccactactatttgtgttggttaatatgggcttcaatatatcacttctacacttactaaagatttcttcagcagttgtatcttctctggcctctacattttcagttccactgtccatctacgccttcaccctaccgttgccgtacatcggtgtggcgtctactctccctcctggctttgttgcaggcgccgtgttgggggatgccagtacagtttctagacgggacaagaaccagggagaaaagaaggagcatcatcacgacggggatgaagagcaagaaatggcggcggcggcggtagcggcggcaacaccaattggggagccacaaggaggatcaccgccgccgccgccatcaacatcatcaggacgctcttgtccatcttggcctcccctgcagctgttgactggactgcctcctccggccggaggctcaccggttgctccactctccggtgttcatccgtcgacttgtgggtcccttctctgggaggtcgaggattcaggtggtccccgggggtgcggcagggaggccatgaccccgacaagcatcgggtga

SEQ ID NO: 26 - protein, 276 aa, Arabidopsis thaliana
MLSVPKSPFLIVGILEALAAAAGMAAAANLSGPSTTVLSQRKPNTRMYSCSSRCNRQCGKVRSAFVLIFCGSGAAHSLNEAGVLWILLMVLSFLLQGAGTVLKEVIFIDSQRRLKGASLDLFIVNSYGSAFQAICIALLLPFLSKLWGIPFNQLGTYLKDGAVCFLNNGTITKGCDGAPFLPLLFVIMNIGYNIALLRLLKISSAVVSCLASTVSVPIAVFLFTMPLPYLGVASSLPKGFMGGTIILVLGMILYSWTPHGANSSHTDSVIPSPPPT

SEQ ID NO: 27 - DNA, 831 bp, Arabidopsis thaliana
atgctctcggtgccaaaatctccatttttaattgttggcatcttagaggctctagctgcagctgctggcatggcagccgcagcgaatctttcggggccatcaacgacagttttatctcagcgtaaaccaaatactcggatgtactcttgtagctctcggtgtaatcgtcagtgtggcaaggttagatctgcttttgtacttatcttttgtggatcaggtgctgctcattcattgaatgaagccggagtattatggattcttctaatggttctctctttcttgcttcaaggagcaggtacagtattgaaggaagtcatctttatagatagccaaagacgattaaagggtgcttcactcgatctatttatagtaaattcatatggttcagctttccaagccatctgcattgcgttgcttcttccatttctttcaaaactttggggcataccgtttaaccaactcggtacctacctaaaagatggtgcggtttgttttctcaacaatgggacaataacaaaaggatgtgatggggctccgtttttgcctctgttgtttgtgataatgaacattggctataacattgctcttttaagactcctcaagatttcatccgcggtggtttcatgtcttgcatcaacagtctcagtgccaattgcagtgtttctgttcacaatgccattaccgtaccttggagttgcatcatcactaccaaaagggttcatgggaggaacaatcatacttgtattgggaatgattctttatagttggacaccacacggagctaactcttctcatactgattcagtcattccttcacctcctccaacatag

SEQ ID NO: 28 - protein, 447 aa, Arabidopsis thaliana
  misc_feature (345)..(345): Xaa can be any naturally occurring amino acid
MATTSSDRLIAGLTASIGSIESRYANPAQSVSLICRNQINGAPPIVLRSSRRSRLWLIEAIPPAKSWNGSNDGDEDIKKSDTRNYAIGGTGGHAVAGKDDRTMEIVIAAATTAALGVGNRVLYKLALIPLKQYPFFLAQLSTFGYVAVYFSILYFRYRAGIVTKEMLSVPKLPFLIVGVLESLALAAGMAAASNLSGPSTTVLSQTFLVWQILFSIIFLGRRYRINQILGCTLVAFGVIVSVASGSGAAHSFKDTGILWSLLMVFSFLLQGADTVMKEVIFLDSKKRLKGASLDLFVVNSYGSIFQVICIALLLPFLSKLWGIPFNQLPSYIRDGGACFLNIGSXITGCEGAPLLPVMFVMMNMAYNISLLRLIKISSAVVSSLASTVSVPIAVYCFTLPLPYLGVASTLPRGFVAGTIILVVGMLLYAWTPSTNTSDSIIPSPXST

SEQ ID NO: 29 - DNA, 1344 bp, Arabidopsis thaliana
atggcaacaacgagctccgaccggttaatcgccggtttaacggcgtcgatcggctcaatcgaatctcgttacgcaaatccagctcaatccgtttctctaatctgtcgaaatcaaatcaatggcgcaccgcctatcgtcttgcggtcctctcgaaggtccaggctgtggttaatcgaggcgattcctccggcaaagtcctggaacggatctaacgatggcgacgaagatatcaagaagtcagacactcgtaattacgcgatcggtggaaccggtggtcacgcggtcgccggaaaagatgacagaacgatggagatagtaatagcmgcggcgaccacggcggcgctgggtgtcgggaatcgcgtactgtataagctggctcttattcctctcaagcaatatcccttcttcctcgcgcaactctccacattcgggtatgtagctgtatacttttcgattttgtatttcagataccgagctggaattgttacaaaagagatgctatcagttccaaaacttccatttttaattgttggcgtcttggagtctcttgctctagctgctgggatggcggccgcatcaaatctttctggaccatctactacagttttatctcagacatttcttgtctggcaaattcttttctccattatatttctcgggaggagatatagaataaatcaaatattgggatgtactcttgtagcatttggtgtaatcgtcagtgtagcaagtgggtcgggtgccgctcattcatttaaagatacaggaatactttggagtcttcttatggtgttttcttttctgcttcaaggagcagatacagtaatgaaggaagtcatctttctagatagcaaaaagcgattgaagggtgcttcacttgatttgtttgttgtaaactcatatggttcaattttccaagtcatatgcattgcgttgcttcttccttttctttcaaagctttggggcataccgtttaaccaactaccgagctatatcagagacgggggtgcttgctttctaaatattggttckrgaataacaggatgcgaaggggctccccttcttcctgtaatgtttgtcatgatgaacatggcttataacatctctcttctacgcctcatcaagatttcatctgcagttgtgtcgtctctggcatccaccgtttcagtgcccatagcggtgtactgtttcacgcttcccctaccataccttggggtggcatctacactaccaagggggtttgttgcaggcacaatcatccttgtagtcggcatgcttctatatgcttggacaccatctactaacacttccgattcaattatcccctcaccccbatccacatag

(19) United States
(12) Patent Application Publication
(10) Pub. No.: US 2010/0011463 A1
(43) Pub. Date: Jan. 14, 2010

(54) Compositions and Methods Using RNA Interference for Control of Nematodes

(75) Inventor: Aaron Wiig, Chapel Hill, NC (US)

Correspondence Address: BASF CORPORATION, CARL-BOSCH-STRASSE 38, LUDWIGSHAFEN, D67056, DE

(73) Assignee: BASF Plant Science GmbH, Ludwigshafen (DE)

(21) Appl. No.: 12/523,202

(22) PCT Filed: Feb. 4, 2008
(86) PCT No.: PCT/EP2008/051326

§ 371 Date: Jul. 15, 2009

Related U.S. Application Data
(60) Provisional application No. 60/899,739, filed on Feb. 6, 2007.

(51) Int. Cl. A01H 1/00; C12N 15/63; C07H 21/02; A01H 5/00

(52) U.S. Cl. 800/278; 435/320.1; 536/23.1; 800/298

(57) ABSTRACT

The present invention concerns double stranded RNA compositions and transgenic plants capable of inhibiting expression of genes essential to establishing or maintaining nematode infestation in a plant, and methods associated therewith. Specifically, the invention relates to the use of RNA interference to inhibit expression of a target plant gene, which is a 50657480 gene or a homolog thereof, and relates to the generation of plants that have increased resistance to parasitic nematodes.

### CROSS REFERENCE TO RELATED APPLICATIONS

This application claims the priority benefit of U.S. Provisional Application Ser. No. 60/899,739 filed Feb. 6, 2007.

### FIELD OF THE INVENTION

The field of this invention is the control of nematodes, in particular the control of soybean cyst nematodes. The invention also relates to the introduction of genetic material into plants that are susceptible to nematodes in order to increase resistance to nematodes.

### BACKGROUND OF THE INVENTION

Nematodes are microscopic wormlike animals that feed on the roots, leaves, and stems of more than 2,000 row crops, vegetables, fruits, and ornamental plants, causing an estimated $100 billion crop loss worldwide. One common type of nematode is the root-knot nematode (RKN), whose feeding causes the characteristic galls on roots. Other root-feeding nematodes are the cyst- and lesion-types, which are more host specific.

Nematodes are present throughout the United States, but are mostly a problem in warm, humid areas of the South and West, and in sandy soils. Soybean cyst nematode (SCN), Heterodera glycines, was first discovered in the United States in North Carolina in 1954. It is the most serious pest of soybean plants. Some areas are so heavily infested by SCN that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. However, nematodes, including SCN, can cause significant yield loss without obvious above-ground symptoms. In addition, roots infected with SCN are dwarfed or stunted. Nematode infestation can decrease the number of nitrogen-fixing nodules on the roots, and may make the roots more susceptible to attacks by other soil-borne plant pathogens.

The nematode life cycle has three major stages: egg, juvenile, and adult. The life cycle varies between species of nematodes. For example, the SCN life cycle can usually be completed in 24 to 30 days under optimum conditions whereas other species can take as long as a year, or longer, to complete the life cycle. When temperature and moisture levels become adequate in the spring, worm-shaped juveniles hatch from eggs in the soil. These juveniles are the only life stage of the nematode that can infect soybean roots.

The life cycle of SCN has been the subject of many studies and therefore can be used as an example for understanding a nematode life cycle. After penetrating the soybean roots, SCN juveniles move through the root until they contact vascular tissue, where they stop and start to feed. The nematode injects secretions that modify certain root cells and transform them into specialized feeding sites. The root cells are morphologically transformed into large multinucleate syncytia (or giant cells in the case of RKN), which are used as a source of nutrients for the nematodes. The actively feeding nematodes thus steal essential nutrients from the plant resulting in yield loss. As the nematodes feed, they swell and eventually female nematodes become so large that they break through the root tissue and are exposed on the surface of the root.

After a period of feeding, male SCN nematodes, which are not swollen as adults, migrate out of the root into the soil and fertilize the lemon-shaped adult females. The males then die, while the females remain attached to the root system and continue to feed. The eggs in the swollen females begin developing, initially in a mass or egg sac outside the body, then later within the body cavity. Eventually the entire body cavity of the adult female is filled with eggs, and the female nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the cysts for several years.

A nematode can move through the soil only a few inches per year on its own power. However, nematode infestation can be spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when contaminated seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties.

Methods have been proposed for the genetic transformation of plants in order to confer increased resistance to plant parasitic nematodes. U.S. Pat. Nos. 5,589,622 and 5,824,876 are directed to the identification of plant genes expressed specifically in or adjacent to the feeding site of the plant after attachment by the nematode. The promoters of these plant target genes can then be used to direct the specific expression of detrimental proteins or enzymes, or the expression of antisense RNA to the target gene or to general cellular genes. The plant promoters may also be used to confer nematode resistance specifically at the feeding site by transforming the plant with a construct comprising the promoter of the plant target gene linked to a gene whose product induces lethality in the nematode after ingestion.

Recently, RNA interference (RNAi), also referred to as gene silencing, has been proposed as a method for controlling nematodes. When double-stranded RNA (dsRNA) corresponding essentially to the sequence of a target gene or mRNA is introduced into a cell, expression from the target gene is inhibited (See e.g., U.S. Pat. No. 6,506,559). U.S. Pat. No. 6,506,559 demonstrates the effectiveness of RNAi against known genes in Caenorhabditis elegans, but does not demonstrate the usefulness of RNAi for controlling plant parasitic nematodes.

Use of RNAi to target essential nematode genes has been proposed, for example, in PCT Publication WO 01/96584, WO 01/17654, US 2004/0098761, US 2005/0091713, US 2005/0188438, US 2006/0037101, US 2006/0080749, US 2007/0199100, and US 2007/0250947.

A number of models have been proposed for the action of RNAi. In mammalian systems, dsRNAs larger than 30 nucleotides trigger induction of interferon synthesis and a global shut-down of protein syntheses, in a non-sequence-specific manner. However, U.S. Pat. No. 6,506,559 discloses that in nematodes, the length of the dsRNA corresponding to the target gene sequence may be at least 25, 50, 100, 200, 300, or 400 bases, and that even larger dsRNAs (742 nucleotides, 1033 nucleotides, 785 nucleotides, 531 nucleotides, 576 nucleotides, 651 nucleotides, 1015 nucleotides, 1033 nucleotides, 730 nucleotides, 830 nucleotides, see Table 1) were also effective at inducing RNAi in C. elegans. It is known that when hairpin RNA constructs comprising double stranded regions ranging from 98 to 854 nucleotides were transformed into a number of plant species, the target plant genes were efficiently silenced. There is general agreement that in many organisms, including nematodes and plants, large pieces of dsRNA are cleaved into about 19-24 nucleotide fragments (siRNA) within cells, and that these siRNAs are the actual mediators of the RNAi phenomenon.

Although there have been numerous efforts to use RNAi to control plant parasitic nematodes, to date no transgenic nematode-resistant plant has been deregulated in any country. Accordingly, there continues to be a need to identify safe and effective compositions and methods for the controlling plant parasitic nematodes using RNAi, and for the production of plants having increased resistance to plant parasitic nematodes.

### SUMMARY OF THE INVENTION

The present inventors have discovered a novel plant target gene (“50657480”) which is overexpressed in syncytia induced by infection of soybean roots by SCN. The inventors have further discovered that when expression of gene 50657480 is suppressed in a soybean root model system, the ability of nematodes to infect such roots is decreased.

In a first embodiment, therefore, the invention provides a double stranded RNA (dsRNA) molecule comprising a) a first strand comprising a sequence substantially identical to a portion of a 50657480-like gene or a 50657480-homolog and b) a second strand comprising a sequence substantially complementary to the first strand.

The invention is further embodied in a pool of dsRNA molecules comprising a multiplicity of RNA molecules each comprising a double stranded region having a length of about 19 to 24 nucleotides, wherein said RNA molecules are derived from a polynucleotide being substantially identical to a portion of a 50657480-like gene or a 50657480-homolog.

In another embodiment, the invention provides a transgenic nematode-resistant plant capable of expressing a dsRNA that is substantially identical to a portion of a 50657480-like gene or a 50657480-homolog.

In another embodiment, the invention provides a transgenic plant capable of expressing a pool of dsRNA molecules, wherein each dsRNA molecule comprises a double stranded region having a length of about 19-24 nucleotides, and wherein the RNA molecules are derived from a polynucleotide substantially identical to a portion of a 50657480-like gene or a 50657480-homolog.

In another embodiment, the invention provides a method of making a transgenic plant capable of expressing a pool of dsRNA molecules each of which is substantially identical to a portion of a 50657480-like gene or a 50657480-homolog in a plant, said method comprising the steps of: a) preparing a nucleic acid having a region that is substantially identical to a portion of a 50657480-like gene or a 50657480-homolog, wherein the nucleic acid is able to form a double-stranded transcript of a portion of a 50657480-like gene or a 50657480-homolog once expressed in the plant; b) transforming a recipient plant with said nucleic acid; c) producing one or more transgenic offspring of said recipient plant; and d) selecting the offspring for expression of said transcript.

The invention further provides a method of conferring nematode resistance to a plant, said method comprising the steps of: a) preparing a nucleic acid having a region that is substantially identical to a portion of a 50657480-like gene or a 50657480-homolog, wherein the nucleic acid is able to form a double-stranded transcript of a portion of a 50657480-like gene or a 50657480-homolog once expressed in the plant; b) transforming a recipient plant with said nucleic acid; c) producing one or more transgenic offspring of said recipient plant; and d) selecting the offspring for nematode resistance.

The invention further provides an expression vector comprising a sequence substantially identical to a portion of a 50657480-like gene or a 50657480-homolog.

### BRIEF DESCRIPTION OF THE DRAWINGS

FIGS. 1a-1c: Table describing primers used to generate the dsRNA construct RAW464 and the RACE fragments corresponding to 50657480/

FIG. 2: DNA sequence alignment of RACE sequence variant A (SEQ ID NO:7) with 50657480 cDNA sequence (SEQ ID NO:1)

FIG. 3: Contig consensus sequence (SEQ ID NO:8) of RACE variant A and 50657480 describing the open reading frame in bold letters.

FIG. 4: Table showing representative homologs of the full length amino acid sequence of 50657480 described by SEQ ID NO:10. The table shows SEQ ID NO, sequence type, organism, and GenBank sequence Id for the representative homologs.

FIGS. 5a-5c: Amino acid sequence alignment of the representative homologs of SEQ ID NO:10.

FIG. 6: Matrix table describing the global amino acid percent identity of the identified representative homologs.

FIG. 7: Matrix table describing the global nucleotide percent identity of the DNA sequences of the identified representative homologs.

FIG. 8a to 8i: shows various 21 mers possible in SEQ ID NO:8 by nucleotide position. For example the 21 mer could comprise nucleotides at position 1 to 21, nucleotides at position 2-22, nucleotides at position 3-23, etc. This table can also be used to calculate the 19, 20, 22, 23 or 24-mers by adding or subtracting the appropriate number of nucleotides from each 21 mer.

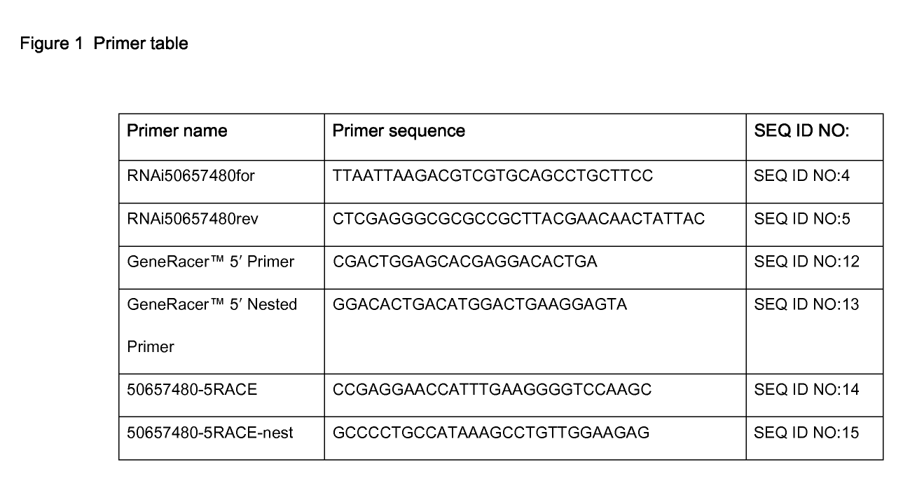

### DETAILED DESCRIPTION OF THE INVENTION

The present invention may be understood more readily by reference to the following detailed description of the preferred embodiments of the invention and the examples included herein. Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5^{th} Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized. It is to be understood that the terminology used herein is for the purpose of describing specific embodiments only and is not intended to be limiting. Throughout this application, various patent and literature publications are referenced. The disclosures of all of these publications and those references cited within those publications in their entireties are hereby incorporated by reference into this application in order to more fully describe the state of the art to which this invention pertains.

In accordance with the invention, a plant is transformed with a nucleic acid or a dsRNA, which specifically inhibits expression of a 50657480 target gene, a 50657480-like gene, or a 50657480 homolog in the plant root that is essential for the development or maintenance of a feeding site, syncytia, or giant cell; ultimately affecting the survival, metamorphosis, or reproduction of the nematode. In a preferred embodiment, inhibition of the 50657480 target gene, a 50657480-like gene, or a 50657480 homolog occurs using dsRNA capable of targeting said gene, which dsRNA has been transformed into an ancestor of the infected plant. Preferably, the nucleic acid sequence expressing the dsRNA is under the transcriptional control of a root specific promoter or a parasitic nematode feeding site-specific promoter or a nematode inducible promoter.

As used herein the terms “target gene”, “50657480 target gene”, “50657480-like gene” and “50657480 gene” refer to genes, which are at least about 50-60%, at least about 60-70%, or at least about 70-75%, 75-80%, 80-85%, 85-90%, or 90-95%, and may also be at least about 96%, 97%, 98%, 99%, or more identical to a polynucleotide comprising the sequence set forth in SEQ ID NO:1, nucleotides 7 to 483 of SEQ ID NO: 1, SEQ ID NO: 7, nucleotides 1 to 1096 of SEQ ID NO:7 or SEQ ID NO:8. Alternatively, suitable 50657480 target genes comprise a polynucleotide that hybridizes under stringent conditions to a polynucleotide comprising the sequence set forth in SEQ ID NO:1 nucleotides 7 to 483 of SEQ ID NO: 1, SEQ ID NO: 7, nucleotides 1 to 1096 of SEQ ID NO:7 or SEQ ID NO:8. The term “50657480 homolog” encompasses genes or sequences, which can be identified by using a part or the full length of any of the sequences disclosed herein, in particular SEQ ID NO: 8, 9, 17, 19, 21, 23, 25, 27, 29 or SEQ ID NO: 4, 5, 14 or 15.

As used herein, “RNAi” or “RNA interference” refers to the process of sequence-specific post-transcriptional gene silencing in plants, mediated by double-stranded RNA (dsRNA). As used herein, “dsRNA” refers to RNA that is partially or completely double stranded. Double stranded RNA is also referred to as small or short interfering RNA (siRNA), short interfering nucleic acid (siNA), short interfering RNA, micro-RNA (miRNA), and the like. In the RNAi process, dsRNA comprising a first strand that is substantially identical to a portion of a target gene and a second strand that is complementary to the first strand is introduced into a plant. After introduction into the plant, the target gene-specific dsRNA is processed into relatively small fragments (siRNAs) and can subsequently become distributed throughout the plant, leading to a loss-of-function mutation having a phenotype that, over the period of a generation, may come to closely resemble the phenotype arising from a complete or partial deletion of the target gene. Alternatively, the target gene-specific dsRNA is operably associated with a regulatory element or promoter that results in expression of the dsRNA in a tissue, temporal, spatial or inducible manner and may further be processed into relatively small fragments by a plant cell containing the RNAi processing machinery, and the loss-of-function phenotype is obtained. Also, the regulatory element or promoter may direct expression preferentially to the roots or syncytia or giant cell where the dsRNA may be expressed either constitutively in those tissues or upon induction by the feeding of the nematode or juvenile nematode, such as J2 nematodes.

As used herein, taking into consideration the substitution of uracil for thymine when comparing RNA and DNA sequences, the term “substantially identical” as applied to dsRNA means that the nucleotide sequence of one strand of the dsRNA is at least 80%-90% identical to 20 or more contiguous nucleotides of the target gene, more preferably, at least 90-95%, identical to 20 or more contiguous nucleotides of the target gene, and most preferably at least 95%, 96%, 97%, 98% or 99% identical or absolutely identical to 20 or more contiguous nucleotides of the target gene. 20 or more contiguous nucleotides means a portion, being at least about 20, 21, 22, 23, 24, 25, 50, 100, 200, 300, 400, 500, 1000, 1500, or 2000 bases or up to the full length of the target gene.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. As used herein, the term “substantially complementary” means that two nucleic acid sequences are complementary over at least 80% of their nucleotides. Preferably, the two nucleic acid sequences are complementary over at least 85%, 90%, 95%, 96%, 97%, 98%, 99% or more of their nucleotides. Alternatively, “substantially complementary” means that two nucleic acid sequences can hybridize under high stringency conditions. As used herein, the term “substantially identical” or “corresponding to” means that two nucleic acid sequences have at least 80% sequence identity. Preferably, the two nucleic acid sequences have at least 85%, 90%, 95%, 96%, 97%, 98%, 99% or 100% of sequence identity.

Also as used herein, the terms “nucleic acid” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “control,” when used in the context of an infection, refers to the reduction or prevention of an infection. Reducing or preventing an infection by a nematode will cause a plant to have increased resistance to the nematode, however, such increased resistance does not imply that the plant necessarily has 100% resistance to infection. In preferred embodiments, the resistance to infection by a nematode in a resistant plant is greater than 10%, 20%, 30%, 40%, 50%, 60%, 70%, 80%, 90%, or 95% in comparison to a wild type plant that is not resistant to nematodes. Preferably the wild type plant is a plant of a similar, more preferably identical genotype as the plant having increased resistance to the nematode, except for the gene responsible for the increased resistance to the nematode. The plant's resistance to infection by the nematode may be due to the death, sterility, arrest in development, or impaired mobility of the nematode upon exposure to the plant comprising dsRNA specific to a gene essential for development or maintenance of a functional feeding site, syncytia, or giant cell. The term “resistant to nematode infection” or “a plant having nematode resistance” as used herein refers to the ability of a plant, as compared to a wild type plant, to avoid infection by nematodes, to kill nematodes or to hamper, reduce or stop the development, growth or multiplication of nematodes. This might be achieved by an active process, e.g. by producing a substance detrimental to the nematode, or by a passive process, like having a reduced nutritional value for the nematode or not developing structures induced by the nematode feeding site like syncytia or giant cells. The level of nematode resistance of a plant can be determined in various ways, e.g. by counting the nematodes being able to establish parasitism on that plant, or measuring development times of nematodes, proportion of male and female nematodes or, for cyst nematodes, counting the number of cysts or nematode eggs produced on roots of an infected plant or plant assay system.

The term “plant” is intended to encompass plants at any stage of maturity or development, as well as any tissues or organs (plant parts) taken or derived from any such plant unless otherwise clearly indicated by context. Plant parts include, but are not limited to, stems, roots, flowers, ovules, stamens, seeds, leaves, embryos, meristematic regions, callus tissue, anther cultures, gametophytes, sporophytes, pollen, microspores, protoplasts, hairy root cultures, and the like. The present invention also includes seeds produced by the plants of the present invention. In one embodiment, the seeds are true breeding for an increased resistance to nematode infection as compared to a wild-type variety of the plant seed. As used herein, a “plant cell” includes, but is not limited to, a protoplast, gamete producing cell, and a cell that regenerates into a whole plant. Tissue culture of various tissues of plants and regeneration of plants therefrom is well known in the art and is widely published.

As used herein, the term “transgenic” refers to any plant, plant cell, callus, plant tissue, or plant part that contains all or part of at least one recombinant polynucleotide. In many cases, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

As used herein, the term “amount sufficient to inhibit expression” refers to a concentration or amount of the dsRNA that is sufficient to reduce levels or stability of mRNA or protein produced from a target gene in a plant. As used herein, “inhibiting expression” refers to the absence or observable decrease in the level of protein and/or mRNA product from a target gene. Inhibition of target gene expression may be lethal to the parasitic nematode either directly or indirectly through modification or eradication of the feeding site, syncytia, or giant cell, or such inhibition may delay or prevent entry into a particular developmental step (e.g., metamorphosis), if access to a fully functional feeding site, syncytia, or giant cell is associated with a particular stage of the parasitic nematode's life cycle. The consequences of inhibition can be confirmed by examination of the plant root for reduction or elimination of cysts or other properties of the nematode or nematode infestation (as presented below in Example 2).

The dsRNA molecule of the invention comprises a first strand that is substantially identical to at least a portion of the 50657480 target gene, the 50657480-like gene, or 50657480 homolog. Preferably the portion of the gene is the full length of the 50657480 target gene as set forth in SEQ ID NO:8, or of the 50657480-like genes and 50657480 homologs as set forth in SEQ ID NOs:17, 19, 21, 23, 25, 27 or 29. More preferably, the dsRNA of the invention comprises a first strand that is substantially identical to from about 19 to about 477 consecutive nucleotides of a sequence selected from the group consisting of: a) a polynucleotide comprising a sequence as set forth in SEQ ID NO:1, nucleotides 7 to 483 of SEQ ID NO: 1, SEQ ID NO: 7, nucleotides 1 to 1096 of SEQ ID NO:7 or SEQ ID NO:8; b) a polynucleotide comprising a sequence having at least 80% sequence identity to SEQ ID NO.1, nucleotides 7 to 483 of SEQ ID NO: 1, SEQ ID NO: 7, nucleotides 1 to 1096 of SEQ ID NO:7 or SEQ ID NO:8; c) a polynucleotide that hybridizes under stringent conditions to a polynucleotide comprising a sequence as set forth in SEQ ID NO:1, nucleotides 7 to 483 of SEQ ID NO: 1, SEQ ID NO: 7, nucleotides 1 to 1096 of SEQ ID NO:7 or SEQ ID NO:8 d) a polynucleotide being obtainable with primers having the sequence as set forth in SEQ ID NO: 4, 5, 14, or 15, e) a polynucleotide comprising a sequence having at least 50% sequence identity to a polynucleotide coding for a nucleotide sequence as set forth in SEQ ID NO: 9, 17, 19, 21, 23, 25, 27 or 29, f) a polynucleotide comprising a sequence having at least 40% sequence identity to a polynucleotide coding for an amino acid sequence as set forth in SEQ ID NO:10, 16, 18, 20, 22, 24, 26 or 28. The dsRNA of the invention further comprises a second strand that is substantially identical to the first strand. The dsRNA of the invention, can be prepared by standard synthetic techniques, e.g., using an automated DNA synthesizer.

Additional 50657480-like genes and 50657480 homologs can be identified with techniques known in the art, such like, but not excluding others, hybridization, RT-PCR, PCR, and the like. For example. 50657480-like genes and 50657480 homologs are obtainable with primers having the sequence as set forth in SEQ ID NO: 4, 5, 12, 13, 14, or 15. 50657480 homologs have at least 50%, 60%, 70, 80%, 90%, 95%, 96%, 97%, 98%, 99% sequence identity to a polynucleotide coding for a nucleotide sequence as set forth in SEQ ID NO: 9, 17, 19, 21, 23, 25, 27 or 29, or have at least 40%, 50%, 60%, 70%, 80%, 90%, 95%, 96%, 97%, 98%, 99% sequence identity to a polynucleotide coding for an amino acid sequence as set forth in SEQ ID NO:10, 16, 18, 20, 22, 24, 26 or 28. Preferably they have at least 50%, 60%, 70, 80%, 90%, 95%, 96%, 97%, 98%, 99% sequence identity to a polynucleotide coding for a nucleotide sequence as set forth in SEQ ID NO: 9, or have at least 40%, 50%, 60%, 70%, 80%, 90%, 95%, 96%, 97%, 98%, 99% sequence identity to a polynucleotide coding for an amino acid sequence as set forth in SEQ ID NO:10. Also preferred are 50657480-like genes and 50657480 homologs having at least 90%, 95%, 96%, 97%, 98%, 99% sequence identity to a polynucleotide coding for a nucleotide sequence as set forth in SEQ ID NO: 9, 17, 19, 21, 23, 25, 27 or 29, or have at least 90%, 95%, 96%, 97%, 98%, 99% sequence identity to a polynucleotide coding for an amino acid sequence as set forth in SEQ ID NO:10, 16, 18, 20, 22, 24, 26 or 28.

For example, a nucleic acid molecule coding for a 50657480-like genes or 50657480 homolog can be isolated from a polynucleotide derived from a plant that hybridizes under stringent conditions to a nucleotide sequence of SEQ ID NO:1, SEQ ID NO: 7 or SEQ ID NO:8. Such a polynucleotide can be isolated from plant tissue cDNA libraries. Alternatively, mRNA can be isolated from plant cells (e.g., by the guanidinium-thiocyanate extraction procedure of Chirgwin et al., 1979, Biochemistry 18:5294-5299), and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon the nucleotide sequence shown in SEQ ID NO:1, SEQ ID NO:7 and SEQ ID NO:8. Nucleic acid molecules corresponding to the plant target genes of the invention can be amplified using cDNA or, alternatively, genomic DNA, as a template and appropriate oligonucleotide primers according to standard PCR amplification techniques. The nucleic acid molecules so amplified can be cloned into appropriate vectors and characterized by DNA sequence analysis. The nucleic acid sequences determined from the cloning of the genes from soybean allow for the generation of probes and primers designed for use in identifying and/or cloning 50657480-like genes and 50657480 homologs in other cell types and organisms, as well as homologs from other plant species. E.g. primers having the sequence as set forth in SEQ ID NO: 4, 5, 12, 13, 14, or 15 can be used in identifying and/or cloning 50657480-like genes and 50657480 homologs.

Such primers can also be used to clone variants of 50657480-like genes and 50657480 homologs. Variants are usually sequence variants having at least 95%, 96%, 97%, 98% or 99% sequence identity to a nucleotide sequence or an amino acid sequence as set forth in SEQ ID NO: 8, 9 or 10. Preferably such variants are obtained from plants of the family Fabaceae, in particular from the genus Glycine.

As discussed above, fragments of dsRNA larger than about 19-24 nucleotides in length are cleaved intracellularly by nematodes and plants to siRNAs of about 19-24 nucleotides in length, and these siRNAs are the actual mediators of the RNAi phenomenon. Thus the dsRNA of the present invention may range in length from about 19 nucleotides up to the whole length of the 50657480-like gene or a 50657480-homolog. Preferably, the dsRNA of the invention has a length from about 21 nucleotides to about 600 nucleotides. More preferably, the dsRNA of the invention has a length from about 21 nucleotides to about 500 nucleotides, or from about 21 nucleotides to about 400 nucleotides.

When dsRNA of the invention has a length longer than about 21 nucleotides, for example from about 50 nucleotides to about 1000 nucleotides, it will be cleaved randomly to dsRNAs of about 21 nucleotides within the plant or parasitic nematode cell, the siRNAs. The cleavage of a longer dsRNA of the invention will yield a pool of about 21 mer dsRNAs (ranging from about 19 mers to about 24 mers), derived from the longer dsRNA. This pool of about 21 mer dsRNAs is also encompassed within the scope of the present invention, whether generated intracellularly within the plant or nematode or synthetically using known methods of oligonucleotide synthesis.

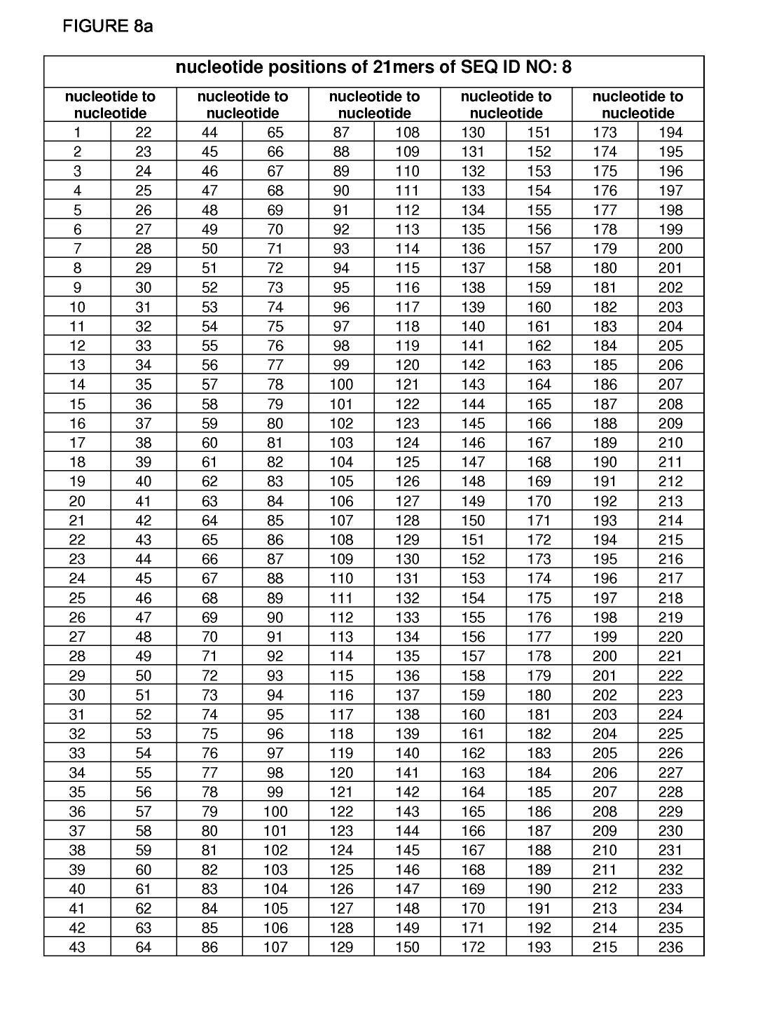

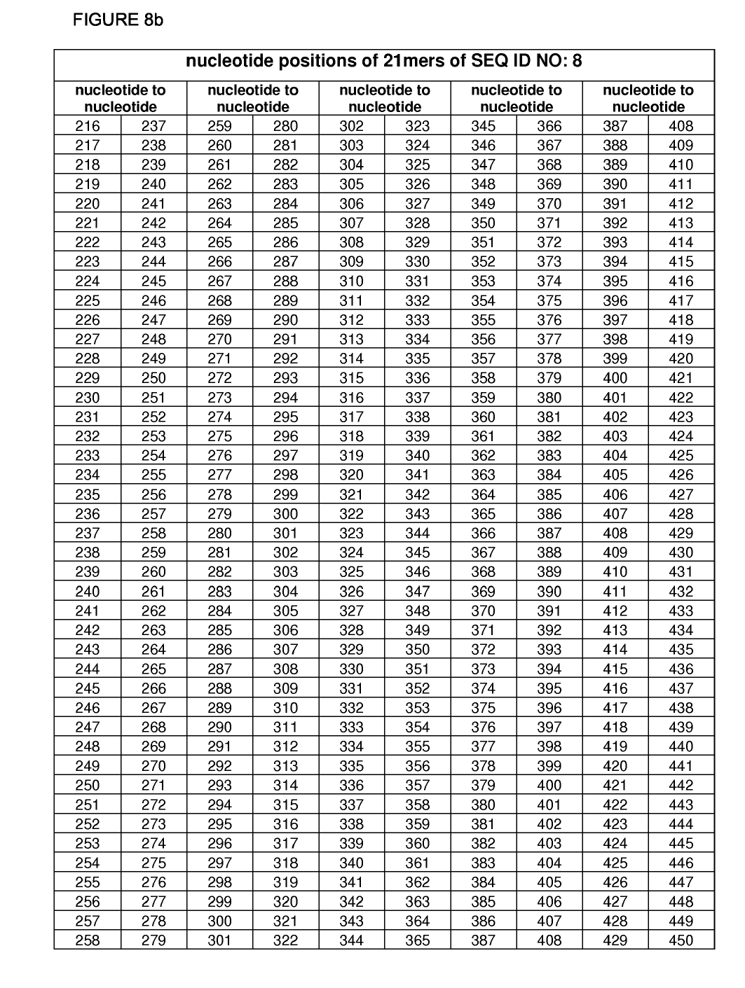

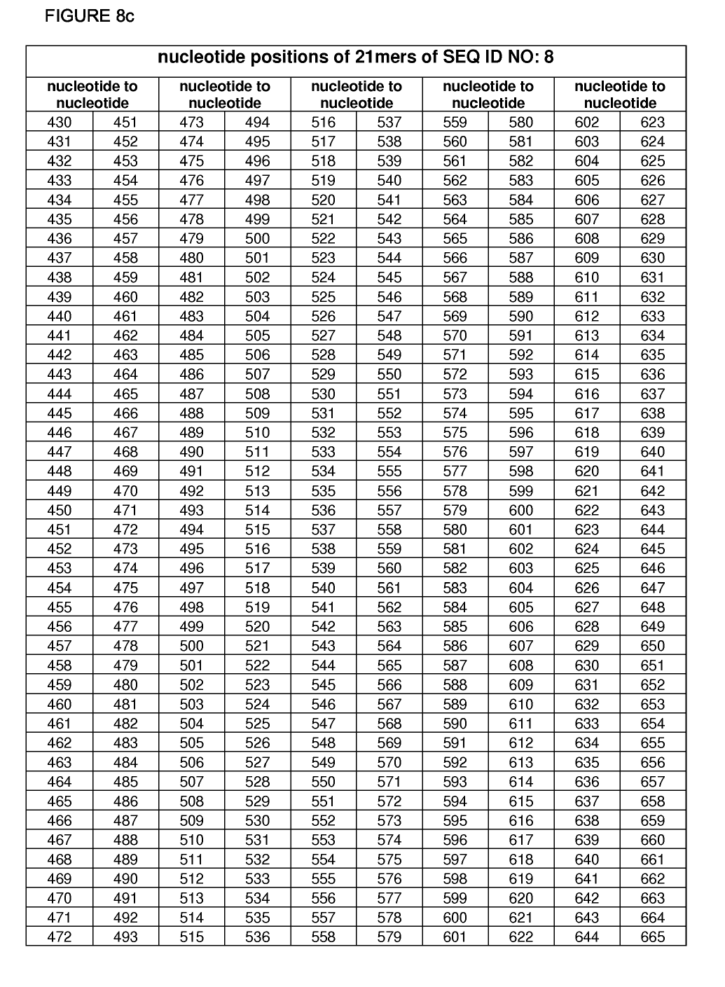

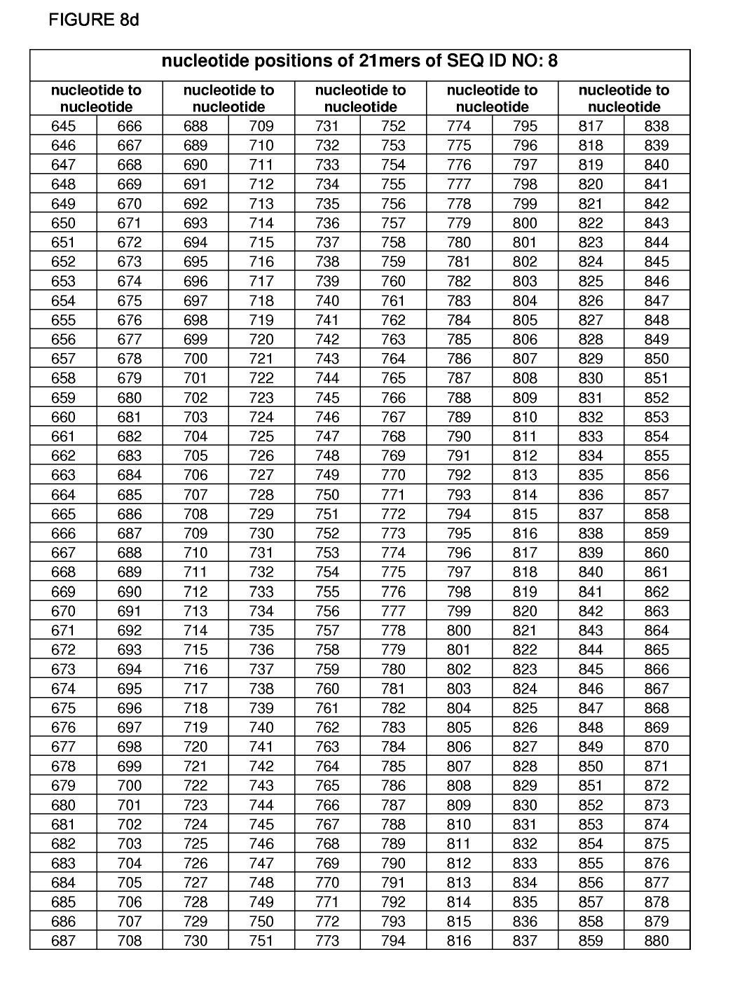

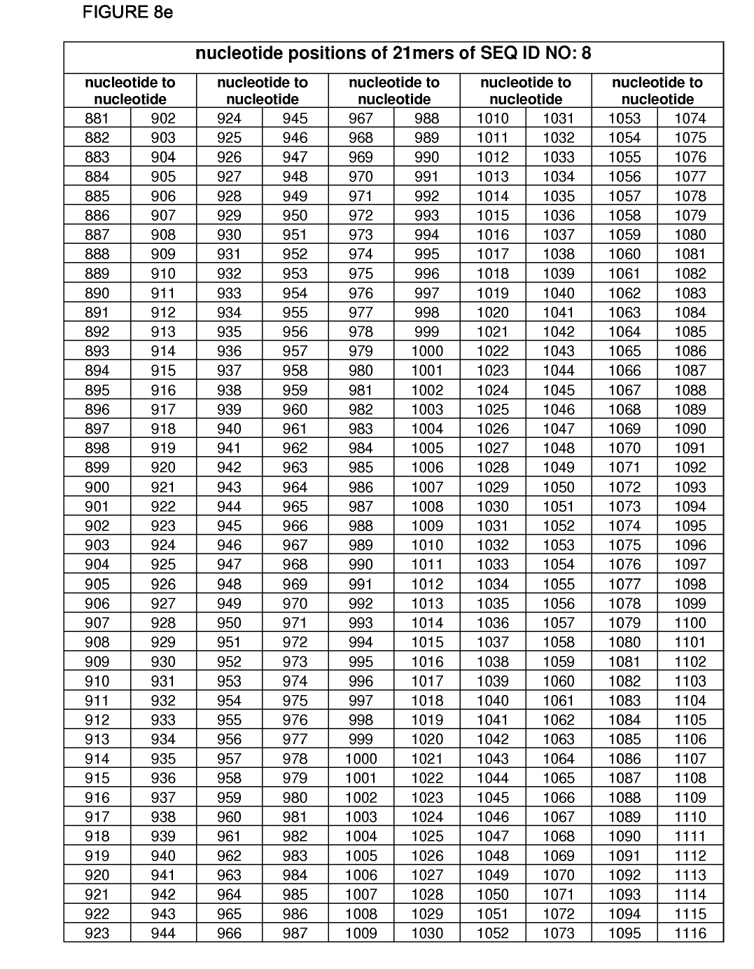

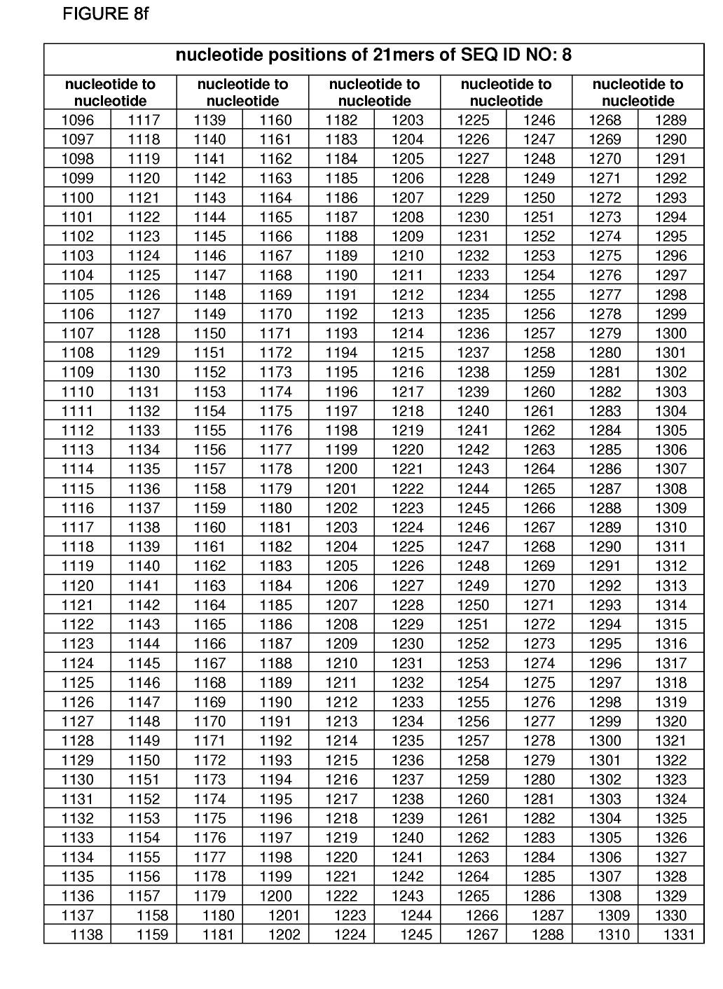

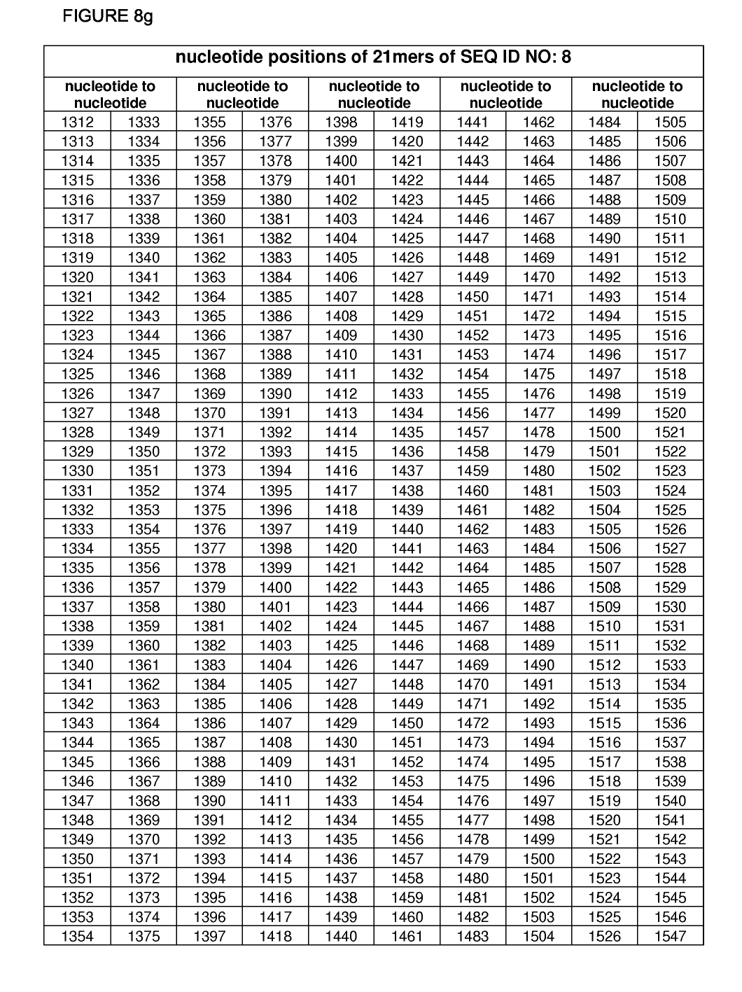

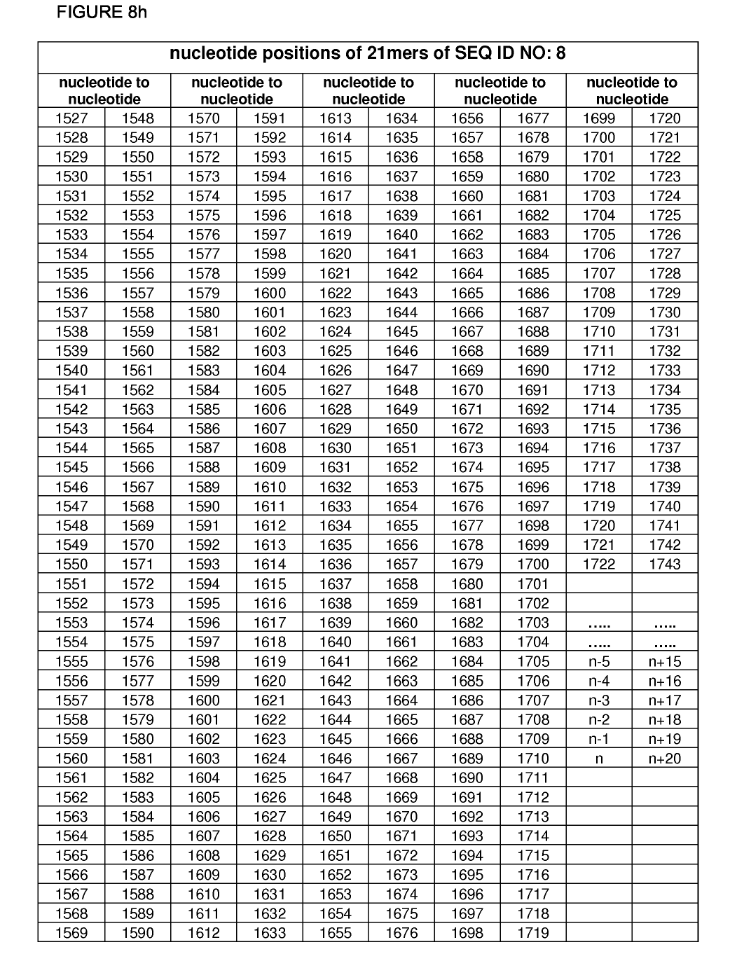

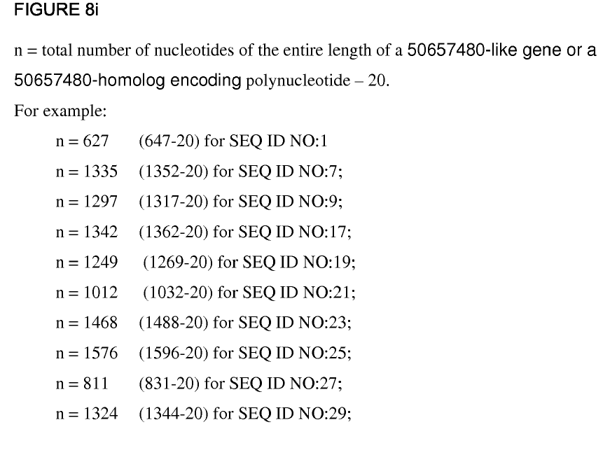

The dsRNAs or siRNAs of the invention have sequences corresponding to fragments of about 19-24 contiguous nucleotides across the entire sequence of the 50657480-like gene or the 50657480-homolog. FIGS. 8a-8e set forth exemplary 21-mers derived from SEQ ID NO:8. In a similar manner, 19-20, 22, 23, and 24-mers derived from SEQ ID NO:8 are encompassed by the present invention.

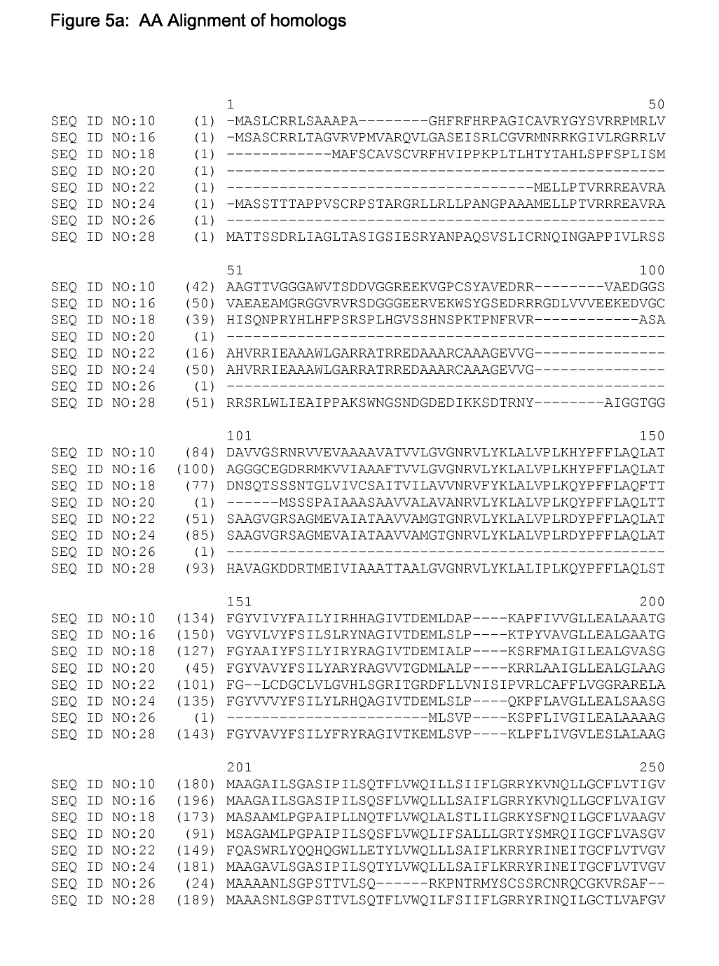

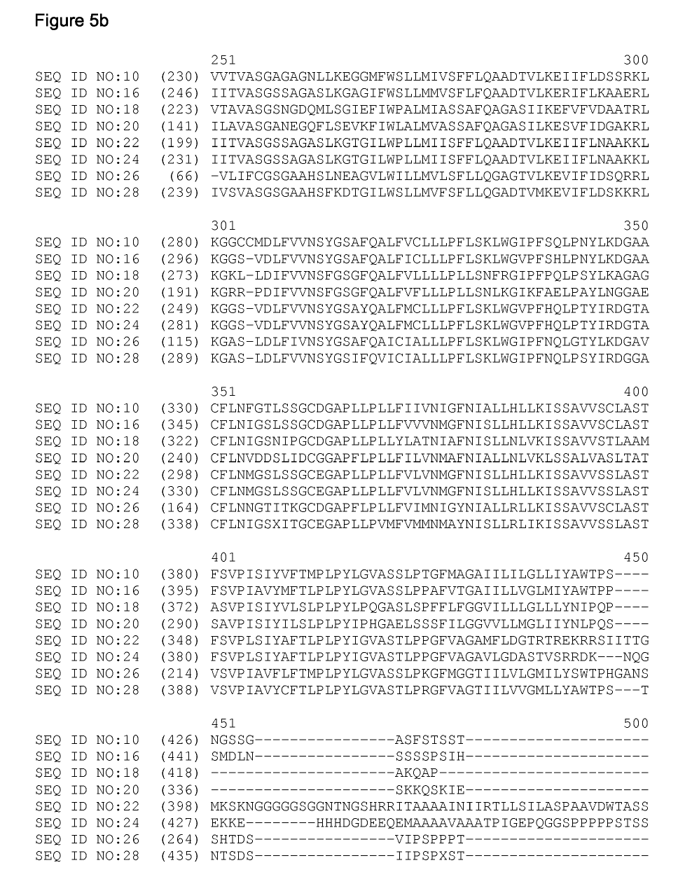

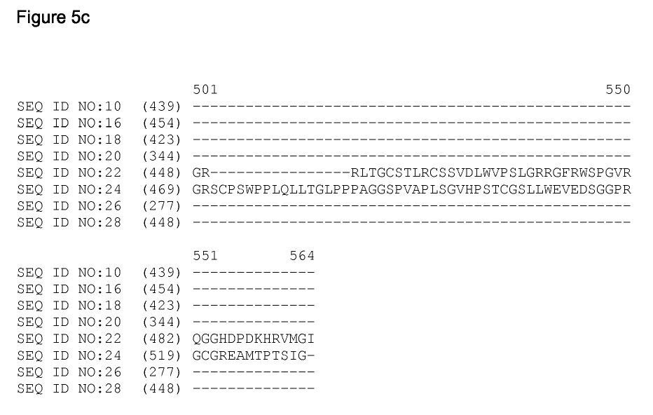

The invention is additionally embodied as a pool of dsRNA molecules derived from a 50657480 gene, a 50657480-like gene, or 50657480 homolog. For example, a pool of siRNA of the invention derived from the 50657480 gene as set forth in SEQ ID NO:1, SEQ ID NO: 7 or SEQ ID NO:8 may comprise a multiplicity of RNA molecules which are selected from the group consisting of oligonucleotides substantially identical to the 21 mer nucleotides of SEQ ID NO:8 as disclosed in FIGS. 8a-8e or any 50657480-like gene or a 50657480-homolog. A pool of siRNA of the invention derived from the 50657480-like gene or the 50657480-homolog e.g. of SEQ ID NO:1, SEQ ID NO: 7 or SEQ ID NO:8 may also comprise any combination of the specific RNA molecules having any of the 21 contiguous nucleotide sequences derived from SEQ ID NO:8 as set forth in FIGS. 8a-8e. The table of FIGS. 8a-8e can also be used to calculate various 19, 20, 22, 23 or 24-mers or start and end of a portion of 50657480-like gene or a 50657480-homolog. Which 19, 20, 22, 23 or 24-mers or portion is the best to choose for a particular plant can be determined with the information given in FIGS. 5, 6 and 7. The 19, 20, 22, 23 or 24-mers or portion having the highest sequence identity to a particular 50657480-like gene or a 50657480-homolog of a particular plant or showing a high degree of sequence conservation in 50657480-like genes or a 50657480-homologs is the most preferred 19, 20, 22, 23 or 24-mer or portion.

A dsRNA comprising a nucleotide sequence identical to a portion of the 50657480 gene, 50657480-like gene or 50657480 homolog is preferred for inhibition. As disclosed herein, 100% sequence identity between the RNA and the 50657480 gene, 50657480-like gene or 50657480 homolog is preferred, but not required to practice the present invention. One of skill in the art will recognize that the siRNA can have a mismatch with the target gene of at least 1, 2, or more nucleotides. Further, these mismatches are intended to be included in the present invention. For example, it is contemplated in the present invention that the 21 mer dsRNA sequences exemplified in FIGS. 8a-8e may contain an addition, deletion or substitution of 1, 2, or more nucleotides and the resulting sequence still interferes with the function of the 50657480 gene, 50657480-like gene or 50657480 homolog. Thus, the invention has the advantage of being able to tolerate sequence variations that might be expected due to gene manipulation or synthesis, genetic mutation, strain polymorphism, or evolutionary divergence.

The degree of sequence identity between the dsRNA and the 50657480 gene, 50657480-like gene or 50657480 homolog may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 80% sequence identity, 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript under stringent conditions (e.g., 400 mM NaCl, 40 mM PIPES pH 6.4, 1 mM EDTA, 60° C. hybridization for 12-16 hours; followed by washing at 65° C. with 0.1% SDS and 0.1% SSC for about 15-60 minutes). The length of the portion or the substantially identical double-stranded nucleotide sequences may be at least about 19, 20, 21, 22, 23, 24, 25, 50, 100, 200, 300, 400, 500, 1000, 1500, or 2000 bases or up to the full length of the gene. In a preferred embodiment, the length of the portion is approximately from about 19 to about 500 nucleotides in length. In another embodiment the portion is from about 50 to about 700 nucleotides in length, in a more preferred embodiment the portion if from about 100 to about 600 nucleotides in length, in an even more preferred embodiment the portion is from about 200 to 500 nucleotides in length. In a further embodiment the portion consists of from about 19 nucleotides to 25% of the whole length of the target gene, more preferred from 25% to 50% even more preferred from 50% to 75% and most preferred 75% to 100% of the whole length of the target gene.

The dsRNA of the invention may optionally comprise a single stranded overhang at either or both ends. The double-stranded structure may be formed by a single self-complementary RNA strand (i.e. forming a hairpin loop) or two complementary RNA strands. RNA duplex formation may be initiated either inside or outside the cell. When the dsRNA of the invention forms a hairpin loop, it may optionally comprise an intron, as set forth in US 2003/0180945A1 or a nucleotide spacer, which is a stretch of sequence between the complementary RNA strands to stabilize the hairpin transgene in cells. Methods for making various dsRNA molecules are set forth, for example, in WO 99/53050 and in U.S. Pat. No. 6,506,559. The RNA may be introduced in an amount that allows delivery of at least one copy per cell. Higher doses of double-stranded material may yield more effective inhibition.

In another embodiment, the invention provides an isolated recombinant expression vector comprising a nucleic acid encoding a dsRNA molecule as described above, wherein expression of the vector in a host plant cell results in increased resistance to a parasitic nematode as compared to a wild-type variety of the host plant cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host plant cell into which they are introduced. Other vectors are integrated into the genome of a host plant cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., potato virus X, tobacco rattle virus, and Geminivirus), which serve equivalent functions.

The recombinant expression vectors of the invention comprise a nucleic acid of the invention in a form suitable for expression of the nucleic acid in a host plant cell, which means that the recombinant expression vector includes one or more regulatory sequences, selected on the basis of the host plant cells to be used for expression, which is operatively linked to the nucleic acid sequence to be expressed. With respect to a recombinant expression vector, the terms “operatively linked” and “in operative association” are interchangeable and are intended to mean that the nucleotide sequence of interest is linked to the regulatory sequence(s) in a manner which allows for expression of the nucleotide sequence (e.g., in a host plant cell when the vector is introduced into the host plant cell). The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990) and Gruber and Crosby, in: Methods in Plant Molecular Biology and Biotechnology, Eds. Glick and Thompson, Chapter 7, 89-108, CRC Press: Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of dsRNA desired, etc. The expression vectors of the invention can be introduced into plant host cells to thereby produce dsRNA molecules of the invention encoded by nucleic acids as described herein.

In accordance with the invention, the recombinant expression vector comprises a regulatory sequence, e.g. a promoter, operatively linked to a nucleotide sequence that is a template for one or both strands of the dsRNA molecules of the invention. In one embodiment, the nucleic acid molecule further comprises a promoter flanking either end of the nucleic acid molecule, wherein the promoters drive expression of each individual DNA strand, thereby generating two complementary RNAs that hybridize and form the dsRNA. In another embodiment, the nucleic acid molecule comprises a nucleotide sequence that is transcribed into both strands of the dsRNA on one transcription unit, wherein the sense strand is transcribed from the 5′ end of the transcription unit and the antisense strand is transcribed from the 3′ end, wherein the two strands are separated by about 3 to about 500 base pairs, and wherein after transcription, the RNA transcript folds on itself to form a hairpin. In accordance with the invention, the spacer region in the hairpin transcript may be any DNA fragment.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and be transiently expressed or transiently active. Whether present in an extra-chromosomal non-replicating vector or a vector that is integrated into a chromosome, the polynucleotide preferably resides in a plant expression cassette. A plant expression cassette preferably contains regulatory sequences capable of driving gene expression in plant cells that are operatively linked so that each sequence can fulfill its function, for example, termination of transcription by polyadenylation signals. Preferred polyadenylation signals are those originating from Agrobacterium tumefaciens t-DNA such as the gene 3 known as octopine synthase of the Ti-plasmid pTiACH5 (Gielen et al., 1984, EMBO J. 3:835) or functional equivalents thereof, but also all other terminators functionally active in plants are suitable. As plant gene expression is very often not limited on transcriptional levels, a plant expression cassette preferably contains other operatively linked sequences like translational enhancers such as the overdrive-sequence containing the 5′-untranslated leader sequence from tobacco mosaic virus enhancing the polypeptide per RNA ratio (Gallie et al., 1987, Nucl. Acids Research 15:8693-8711). Examples of plant expression vectors include those detailed in: Becker, D. et al., 1992, New plant binary vectors with selectable markers located proximal to the left border, Plant Mol. Biol. 20:1195-1197; Bevan, M. W., 1984, Binary Agrobacterium vectors for plant transformation, Nucl. Acid. Res. 12:8711-8721; and Vectors for Gene Transfer in Higher Plants; in: Transgenic Plants, Vol. 1, Engineering and Utilization, eds.: Kung and R. Wu, Academic Press, 1993, S. 15-38.

Plant gene expression should be operatively linked to an appropriate promoter conferring gene expression in a temporal-preferred, spatial-preferred, cell type-preferred, and/or tissue-preferred manner. Promoters useful in the expression cassettes of the invention include any promoter that is capable of initiating transcription in a plant cell present in the plant's roots. Such promoters include, but are not limited to those that can be obtained from plants, plant viruses and bacteria that contain genes that are expressed in plants, such as Agrobacterium and Rhizobium. Preferably, the expression cassette of the invention comprises a root-specific promoter, a pathogen inducible promoter or a nematode inducible promoter. More Preferably the nematode inducible promoter is a parasitic nematode feeding site-specific promoter. A parasitic nematode feeding site-specific promoter may be specific for syncytial cells or giant cells or specific for both kinds of cells. A promoter is inducible, if its activity, measured on the amount of RNA produced under control of the promoter, is at least 30%, 40%, 50% preferably at least 60%, 70%, 80%, 90% more preferred at least 100%, 200%, 300% higher in its induced state, than in its un-induced state. A promoter is cell-, tissue- or organ-specific, if its activity, measured on the amount of RNA produced under control of the promoter, is at least 30%, 40%, 50% preferably at least 60%, 70%, 80%, 90% more preferred at least 100%, 200%, 300% higher in a particular cell-type, tissue or organ, then in other cell-types or tissues of the same plant, preferably the other cell-types or tissues are cell types or tissues of the same plant organ, e.g. a root. In the case of organ specific promoters, the promoter activity has to be compared to the promoter activity in other plant organs, e.g. leafs, stems, flowers or seeds.

The promoter may be constitutive, inducible, developmental stage-preferred, cell type-preferred, tissue-preferred or organ-preferred. Constitutive promoters are active under most conditions. Non-limiting examples of constitutive promoters include the CaMV 19S and 35S promoters (Odell et al., 1985, Nature 313:810-812), the sX CaMV 35S promoter (Kay et al., 1987, Science 236:1299-1302), the Sep1 promoter, the rice actin promoter (McElroy et al., 1990, Plant Cell 2:163-171), the Arabidopsis actin promoter, the ubiquitin promoter (Christensen et al., 1989, Plant Molec. Biol. 18:675-689); pEmu (Last et al., 1991, Theor. Appl. Genet. 81:581-588), the figwort mosaic virus 35S promoter, the Smas promoter (Velten et al., 1984, EMBO J. 3:2723-2730), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like. Promoters that express the dsRNA in a cell that is contacted by parasitic nematodes are preferred. Alternatively, the promoter may drive expression of the dsRNA in a plant tissue remote from the site of contact with the nematode, and the dsRNA may then be transported by the plant to a cell that is contacted by the parasitic nematode, in particular cells of or close by feeding sites, e.g. syncytial cells or giant cells.

Inducible promoters are active under certain environmental conditions, such as the presence or absence of a nutrient or metabolite, heat or cold, light, pathogen attack, anaerobic conditions, and the like. For example, the promoters TobRB7, AtRPE, AtPyk10, Gemini19, and AtHMG1 have been shown to be induced by nematodes (for a review of nematode-inducible promoters, see Ann. Rev. Phytopathol. (2002) 40:191-219; see also U.S. Pat. No. 6,593,513). Method for isolating additional promoters, which are inducible by nematodes are set forth in U.S. Pat. Nos. 5,589,622 and 5,824,876. Other inducible promoters include the hsp80 promoter from Brassica, being inducible by heat shock; the PPDK promoter is induced by light; the PR-1 promoter from tobacco, Arabidopsis, and maize are inducible by infection with a pathogen; and the Adh1 promoter is induced by hypoxia and cold stress. Plant gene expression can also be facilitated via an inducible promoter (For review, see Gatz, 1997, Annu. Rev. Plant Physiol. Plant Mol. Biol. 48:89-108). Chemically inducible promoters are especially suitable if time-specific gene expression is desired. Non-limiting examples of such promoters are a salicylic acid inducible promoter (PCT Application No. WO 95/19443), a tetracycline inducible promoter (Gatz et al., 1992, Plant J. 2:397-404) and an ethanol inducible promoter (PCT Application No. WO 93/21334).

Developmental stage-preferred promoters are preferentially expressed at certain stages of development. Tissue and organ preferred promoters include those that are preferentially expressed in certain tissues or organs, such as, but not limited to leaves, roots, seeds, or xylem. Examples of tissue preferred and organ preferred promoters include, but are not limited to fruit-preferred, ovule-preferred, male tissue-preferred, seed-preferred, integument-preferred, tuber-preferred, stalk-preferred, pericarp-preferred, and leaf-preferred, stigma-preferred, pollen-preferred, anther-preferred, a petal-preferred, sepal-preferred, pedicel-preferred, silique-preferred, stem-preferred, root-preferred promoters and the like. Seed preferred promoters are preferentially expressed during seed development and/or germination. For example, seed preferred promoters can be embryo-preferred, endosperm preferred and seed coat-preferred. See Thompson et al., 1989, BioEssays 10:108. Examples of seed preferred promoters include, but are not limited to cellulose synthase (celA), Cim1, gamma-zein, globulin-1, maize 19 kD zein (cZ19B1) and the like.

Other suitable tissue-preferred or organ-preferred promoters include, but are not limited to, the napin-gene promoter from rapeseed (U.S. Pat. No. 5,608,152), the USP-promoter from Vicia faba (Baeumlein et al., 1991, Mol Gen Genet. 225(3):459-67), the oleosin-promoter from Arabidopsis (PCT Application No. WO 98/45461), the phaseolin-promoter from Phaseolus vulgaris (U.S. Pat. No. 5,504,200), the Bce4-promoter from Brassica (PCT Application No. WO 91/13980), or the legumin B4 promoter (LeB4; Baeumlein et al., 1992, Plant Journal, 2(2):233-9), as well as promoters conferring seed specific expression in monocot plants like maize, barley, wheat, rye, rice, etc. Suitable promoters to note are the Ipt2 or Ipt1-gene promoter from barley (PCT Application No. WO 95/15389 and PCT Application No. WO 95/23230) or those described in PCT Application No. WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene, and rye secalin gene).

Other promoters useful in the expression cassettes of the invention include, but are not limited to, the major chlorophyll a/b binding protein promoter, histone promoters, the Ap3 promoter, the β-conglycin promoter, the napin promoter, the soybean lectin promoter, the maize 15 kD zein promoter, the 22 kD zein promoter, the 27 kD zein promoter, the g-zein promoter, the waxy, shrunken 1, shrunken 2, and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), as well as synthetic or other natural promoters

In accordance with the present invention, the expression cassette comprises an expression control sequence operatively linked to a nucleotide sequence that is a template for one or both strands of the dsRNA. The dsRNA template comprises (a) a first stand having a sequence substantially identical to from about 19 to about 500, or up to the full length, consecutive nucleotides of SEQ ID NO:1, SEQ ID NO: 7 or SEQ ID NO:8; and (b) a second strand having a sequence substantially complementary to the first strand. In further embodiments, a promoter flanks either end of the template nucleotide sequence, wherein the promoters drive expression of each individual DNA strand, thereby generating two complementary RNAs that hybridize and form the dsRNA. In alternative embodiments, the nucleotide sequence is transcribed into both strands of the dsRNA on one transcription unit, wherein the sense strand is transcribed from the 5′ end of the transcription unit and the anti-sense strand is transcribed from the 3′ end, wherein the two strands are separated by about 3 to about 500 base pairs, and wherein after transcription, the RNA transcript folds on itself to form a hairpin.

In another embodiment, the vector contains a bidirectional promoter, driving expression of two nucleic acid molecules, whereby one nucleic acid molecule codes for the sequence substantially identical to a portion of a 50657480-like gene or a 50657480-homolog and the other nucleic acid molecule codes for a second sequence being substantially complementary to the first strand and capable of forming a dsRNA, when both sequences are transcribed. A bidirectional promoter is a promoter capable of mediating expression in two directions.

In another embodiment, the vector contains two promoters one mediating transcription of the sequence substantially identical to a portion of a 50657480-like gene or a 50657480-homolog and another promoter mediating transcription of a second sequence being substantially complementary to the first strand and capable of forming a dsRNA, when both sequences are transcribed. The second promoter might be a different promoter.

A different promoter means a promoter having a different activity in regard to cell or tissue specificity, or showing expression on different inducers for example, pathogens, abiotic stress or chemicals. For example, one promoter might be constitutive or tissue specific and another might be tissue specific or inducible by pathogens. In one embodiment one promoter mediates the transcription of one nucleic acid molecule suitable for overexpression of a 50657480 gene, while another promoter mediates tissue- or cell-specific transcription or pathogen inducible expression of the complementary nucleic acid.

The invention is also embodied in a transgenic plant capable of expressing the dsRNA of the invention and thereby inhibiting the 50657480-like genes or 50657480 homolog (target gene) in the roots, feeding site, syncytia and/or giant cell

The plant or transgenic plant may be any plant, such like, but not limited to trees, cut flowers, ornamentals, vegetables or crop plants. The plant may be from a genus selected from the group consisting of Medicago, Lycopersicon, Brassica, Cucumis, Solanum, Juglans, Gossypium, Malus, Vitis, Antirrhinum, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Medicago, Onobrychis, trifolium, Trigonella, Vigna, Citrus, Linum, Geranium, Manihot, Daucus, Raphanus, Sinapis, Atropa, Datura, Hyoscyamus, Nicotiana, Petunia, Digitalis, Majorana, Ciahorium, Lactuca, Bromus, Asparagus, Antirrhinum, Heterocallis, Nemesis, Pelargonium, Panieum, Pennisetum, Ranunculus, Senecio, Salpiglossis, Browaalia, Phaseolus, Avena, and Allium, or the plant may be selected from a genus selected from the group consisting of Arabidopsis, Medicago, Lycopersicon, Brassica, Cucumis, Solanum, Juglans, Gossypium, Malus, Vitis, Antirrhinum, Brachipodium, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Medicago, Onobrychis, trifolium, Trigonella, Vigna, Citrus, Linum, Geranium, Manihot, Daucus, Raphanus, Sinapis, Atropa, Datura, Hyoscyamus, Nicotiana, Petunia, Digitalis, Majorana, Ciahorium, Lactuca, Bromus, Asparagus, Antirrhinum, Heterocallis, Nemesis, Pelargonium, Panicum, Pennisetum, Ranunculus, Senecio, Salpiglossis, Browaalia, Phaseolus, Avena, and Allium. In one embodiment the plant is a monocotyledonous plant or a dicotyledonous plant.

Preferably the plant is a crop plant. Crop plants are all plants, used in agriculture. Accordingly in one embodiment the plant is a monocotyledonous plant, preferably a plant of the family Poaceae, Musaceae, Liliaceae or Bromeliaceae, preferably of the family Poaceae. Accordingly, in yet another embodiment the plant is a Poaceae plant of the genus Zea, Triticum, Oryza, Hordeum, Secale, Avena, Saccharum, Sorghum, Pennisetum, Setaria, Panicum, Eleusine, Miscanthus, Brachypodium, Festuca or Lolium. When the plant is of the genus Zea, the preferred species is Z. mays. When the plant is of the genus Triticum, the preferred species is T. aestivum, T. speltae or T. durum. When the plant is of the genus Oryza, the preferred species is O. sativa. When the plant is of the genus Hordeum, the preferred species is H. vulgare. When the plant is of the genus Secale, the preferred species S. cereale. When the plant is of the genus Avena, the preferred species is A. sativa. When the plant is of the genus Saccarum, the preferred species is S. officinarum. When the plant is of the genus Sorghum, the preferred species is S. vulgare, S. bicolor or S. sudanense. When the plant is of the genus Pennisetum, the preferred species is P. glaucum. When the plant is of the genus Setaria, the preferred species is S. italica. When the plant is of the genus Panicum, the preferred species is P. miliaceum or P. virgatum. When the plant is of the genus Eleusine, the preferred species is E. coracana. When the plant is of the genus Miscanthus, the preferred species is M. sinensis. When the plant is a plant of the genus Festuca, the preferred species is F. arundinaria, F. rubra or F. pratensis. When the plant is of the genus Lolium, the preferred species is L. perenne or L. multiflorum. Alternatively, the plant may be Triticosecale.

Alternatively, in one embodiment the plant is a dicotyledonous plant, preferably a plant of the family Fabaceae, Solanaceae, Brassicaceae, Chenopodiaceae, Asteraceae, Malvaceae, Linacea, Euphorbiaceae, Convolvulaceae Rosaceae, Cucurbitaceae, Theaceae, Rubiaceae, Sterculiaceae or Citrus. In one embodiment the plant is a plant of the family Fabaceae, Solanaceae or Brassicaceae. Accordingly, in one embodiment the plant is of the family Fabaceae, preferably of the genus Glycine, Pisum, Arachis, Cicer, Vicia, Phaseolus, Lupinus, Medicago or Lens. Preferred species of the family Fabaceae are M. truncatula, M, sativa, G. max, P. sativum, A. hypogea, C. arietinum, V. faba, P. vulgaris, Lupinus albus, Lupinus luteus, Lupinus angustifolius or Lens culinaris. More preferred are the species G. max A. hypogea and M. sativa. Most preferred is the species G. max. When the plant is of the family Solanaceae, the preferred genus is Solanum, Lycopersicon, Nicotiana or Capsicum. Preferred species of the family Solanaceae are S. tuberosum, L. esculentum, N. tabaccum or C. chinense. More preferred is S. tuberosum. Accordingly, in one embodiment the plant is of the family Brassicaceae, preferably of the genus Brassica or Raphanus. Preferred species of the family Brassicaceae are the species B. napus, B. oleracea, B. juncea or B. rapa. More preferred is the species B. napus. When the plant is of the family Chenopodiaceae, the preferred genus is Beta and the preferred species is the B. vulgaris. When the plant is of the family Asteraceae, the preferred genus is Helianthus and the preferred species is H. annuus. When the plant is of the family Malvaceae, the preferred genus is Gossypium or Abelmoschus. When the genus is Gossypium, the preferred species is G. hirsutum or G. barbadense and the most preferred species is G. hirsutum. A preferred species of the genus Abelmoschus is the species A. esculentus. When the plant is of the family Linacea, the preferred genus is Linum and the preferred species is L. usitatissimum. When the plant is of the family Euphorbiaceae, the preferred genus is Manihot, Jatropa or Rhizinus and the preferred species are M. esculenta, J. curcas or R. comunis. When the plant is of the family Convolvulaceae, the preferred genus is Ipomea and the preferred species is I. batatas. When the plant is of the family Rosaceae, the preferred genus is Rosa, Malus, Pyrus, Prunus, Rubus, Ribes, Vaccinium or Fragaria and the preferred species is the hybrid Fragaria x ananassa. When the plant is of the family Cucurbitaceae, the preferred genus is Cucumis, Citrullus or Cucurbita and the preferred species is Cucumis sativus, Citrullus lanatus or Cucurbita pepo. When the plant is of the family Theaceae, the preferred genus is Camellia and the preferred species is C. sinensis. When the plant is of the family Rubiaceae, the preferred genus is Coffea and the preferred species is C. arabica or C. canephora. When the plant is of the family Sterculiaceae, the preferred genus is Theobroma and the preferred species is T. cacao. When the plant is of the genus Citrus, the preferred species is C. sinensis, C. limon, C. reticulata, C. maxima and hybrids of Citrus species, or the like. In a preferred embodiment of the invention, the plant is a soybean, a potato or a corn plant.

Suitable methods for transforming or transfecting host cells including plant cells are well known in the art of plant biotechnology. Any method may be used to transform the recombinant expression vector into plant cells to yield the transgenic plants of the invention. General methods for transforming dicotyledenous plants are disclosed, for example, in U.S. Pat. Nos. 4,940,838; 5,464,763, and the like. Methods for transforming specific dicotyledenous plants, for example, cotton, are set forth in U.S. Pat. Nos. 5,004,863; 5,159,135; and 5,846,797. Soybean transformation methods are set forth in U.S. Pat. Nos. 4,992,375; 5,416,011; 5,569,834; 5,824,877; 6,384,301 and in EP 0301749B1 may be used.

Transformation methods may include direct and indirect methods of transformation. Suitable direct methods include polyethylene glycol induced DNA uptake, liposome-mediated transformation (U.S. Pat. No. 4,536,475), biolistic methods using the gene gun (Fromm M E et al., Bio/Technology. 8(9):833-9, 1990; Gordon-Kamm et al. Plant Cell 2:603, 1990), electroporation, incubation of dry embryos in DNA-comprising solution, and microinjection. In the case of these direct transformation methods, the plasmids used need not meet any particular requirements. Simple plasmids, such as those of the pUC series, pBR322, M13 mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch R B et al. (1985) Science 225:1229. The Agrobacterium-mediated transformation is best suited to dicotyledonous plants but has also been adapted to monocotyledonous plants. The transformation of plants by Agrobacteria is described in, for example, White F F, Vectors for Gene Transfer in Higher Plants, Transgenic Plants, Vol. 1, Engineering and Utilization, edited by S. D. Kung and R. Wu, Academic Press, 1993, pp. 15-38; Jenes B et al. Techniques for Gene Transfer, Transgenic Plants, Vol. 1, Engineering and Utilization, edited by S. D. Kung and R. Wu, Academic Press, 1993, pp. 128-143; Potrykus (1991) Annu Rev Plant Physiol Plant Molec Biol 42:205-225.

Transformation may result in transient or stable transformation and expression. Although a nucleotide sequence of the present invention can be inserted into any plant and plant cell falling within these broad classes, it is particularly useful in crop plant cells.

The transgenic plants of the invention may be crossed with similar transgenic plants or with transgenic plants lacking the nucleic acids of the invention or with non-transgenic plants, using known methods of plant breeding, to prepare seeds. Further, the transgenic plant of the present invention may comprise, and/or be crossed to another transgenic plant that comprises one or more nucleic acids, thus creating a “stack” of transgenes in the plant and/or its progeny. The seed is then planted to obtain a crossed fertile transgenic plant comprising the nucleic acid of the invention. The crossed fertile transgenic plant may have the particular expression cassette inherited through a female parent or through a male parent. The second plant may be an inbred plant. The crossed fertile transgenic may be a hybrid. Also included within the present invention are seeds of any of these crossed fertile transgenic plants. The seeds of this invention can be harvested from fertile transgenic plants and be used to grow progeny generations of transformed plants of this invention including hybrid plant lines comprising the DNA construct.

“Gene stacking” can also be accomplished by transferring two or more genes into the cell nucleus by plant transformation. Multiple genes may be introduced into the cell nucleus during transformation either sequentially or in unison. Multiple genes in plants or target pathogen species can be down-regulated by gene silencing mechanisms, specifically RNAi, by using a single transgene targeting multiple linked partial sequences of interest. Stacked, multiple genes under the control of individual promoters can also be over-expressed to attain a desired single or multiple phenotype. Constructs containing gene stacks of both over-expressed genes and silenced targets can also be introduced into plants yielding single or multiple agronomically important phenotypes. In certain embodiments the nucleic acid sequences of the present invention can be stacked with any combination of polynucleotide sequences of interest to create desired phenotypes. The combinations can produce plants with a variety of trait combinations including but not limited to disease resistance, herbicide tolerance, yield enhancement, cold and drought tolerance. These stacked combinations can be created by any method including but not limited to cross breeding plants by conventional methods or by genetic transformation. If the traits are stacked by genetic transformation, the polynucleotide sequences of interest can be combined sequentially or simultaneously in any order. For example if two genes are to be introduced, the two sequences can be contained in separate transformation cassettes or on the same transformation cassette. The expression of the sequences can be driven by the same or different promoters.

In accordance with this embodiment, the transgenic plant of the invention is produced by a method comprising the steps of providing a preparing an expression cassette having a first region that is substantially identical to a portion of a 50657480 gene, a 50657480-like gene or a 50657480 homolog, and a second region which is complementary to the first region, transforming the expression cassette into a plant, and selecting progeny of the transformed plant which express the dsRNA construct of the invention.

The present invention may be used to reduce crop destruction by any plant parasitic nematode. Preferably, the parasitic nematodes belong to nematode families inducing giant or syncytial cells. Nematodes inducing giant or syncytial cells are found in the families Longidoridae, Trichodoridae, Heterodidae, Meloidogynidae, Pratylenchidae or Tylenchulidae. In particular in the families Heterodidae and Meloidogynidae.

Accordingly, parasitic nematodes targeted by the present invention belong to one or more genus selected from the group of Naccobus, Cactodera, Dolichodera, Globodera, Heterodera, Punctodera, Longidorus or Meloidogyne. In a preferred embodiment the parasitic nematodes belong to one or more genus selected from the group of Naccobus, Cactodera, Dolichodera, Globodera, Heterodera, Punctodera or Meloidogyne. In a more preferred embodiment the parasitic nematodes belong to one or more genus selected from the group of Globodera, Heterodera, or Meloidogyne. In an even more preferred embodiment the parasitic nematodes belong to one or both genus selected from the group of Globodera or Heterodera. In another embodiment the parasitic nematodes belong to the genus Meloidogyne.

When the parasitic nematodes are of the genus Globodera, the species are preferably from the group consisting of G. achilleae, G. artemisiae, G. hypolysi, G. mexicana, G. millefolii, G. mali, G. pallida, G. rostochiensis, G. tabacum, and G. virginiae. In another preferred embodiment the parasitic Globodera nematodes includes at least one of the species G. pallida, G. tabacum, or G. rostochiensis. When the parasitic nematodes are of the genus Heterodera, the species may be preferably from the group consisting of H. avenae, H. carotae, H. ciceri, H. cruciferae, H. delvii, H. elachista, H. filipjevi, H. gambiensis, H. glycines, H. goettingiana, H. graduni, H. humuli, H. hordecalis, H. latipons, H. major, H. medicaginis, H. oryzicola, H. pakistanensis, H. rosii, H. sacchari, H. schachtii, H. sorghi, H. trifolii, H. urticae, H. vigni and H. zeae. In another preferred embodiment the parasitic Heterodera nematodes include at least one of the species H. glycines, H. avenae, H. cajani, H. gottingiana, H. trifolii, H. zeae or H. schachtii. In a more preferred embodiment the parasitic nematodes includes at least one of the species H. glycines or H. schachtii. In a most preferred embodiment the parasitic nematode is the species H. glycines.

When the parasitic nematodes are of the genus Meloidogyne, the parasitic nematode may be selected from the group consisting of M. acronea, M. arabica, M. arenaria, M. artiellia, M. brevicauda, M. camelliae, M. chitwoodi, M. cofeicola, M. esigua, M. graminicola, M. hapla, M. incognita, M. indica, M. inornata, M. javanica, M. lini, M. mali, M. microcephala, M. microtyla, M. naasi, M. salasi and M. thamesi. In a preferred embodiment the parasitic nematodes includes at least one of the species M. javanica, M. incognita, M. hapla, M. arenaria or M. chitwoodi.

The present invention also provides a method for inhibiting expression of a 50657480 gene, a 50657480-like gene, or a 50657480 homolog. In accordance with this embodiment, the method comprises the step of administering to the plant a dsRNA of the invention.

The following examples are not intended to limit the scope of the claims to the invention, but are rather intended to be exemplary of certain embodiments. Any variations in the exemplified methods that are within the ordinary level of skill in the art are intended to fall within the scope of the present invention.

### Example 1

### Cloning of 50657480 from Soybean

### Laser Excision of Syncytia

Glycine max cv. Williams 82 was germinated on agar plates for three days and then transferred to germination pouches. One day later, each seedling was inoculated with second stage juveniles (J2) of H. glycines race 3. Six days after inoculation, new root tissue was sliced into 1 cm long pieces, fixed, embedded in a cryomold, and sectioned using known methods. Syncytia cells were identified by their unique morphology of enlarged cell size, thickened cell wall, and dense cytoplasm and dissected into RNA extraction buffer using a PALM microscope (P.A.L.M. Microlaser Technologies GmbH, Bernried, Germany).

Total cellular RNA was extracted, amplified, and fluorescently labeled using known methods. As controls, total RNA was isolated from both “non-syncytia” and untreated control roots subjected to the same RNA amplification process. The amplified RNA was hybridized to proprietary soybean cDNA arrays.

As demonstrated in Table 2, Soybean cDNA clone 50657480 was identified as being up-regulated in syncytia of SCN-infected soybean roots. The amino acid sequence of soybean cDNA clone 50657480 (SEQ ID NO:1) is described as SEQ ID NO: 3. The 50657480 cDNA sequence (SEQ ID NO:1) was determined not to be full-length as there no ATG start codon.

TABLE 2

| Gene | Syncytia #1 | Syncytia #2 | | Control |
|---|---|---|---|---|
| Name | (N)^{¶} | (N) | Non-Syncytia | Roots |
| 50657480^{§} | 299 ± 47 (4) | 369 ± 57 (5) | not detected | not detected |

### Example 2

### Generation of Transgenic Soybean Hairy-Root and Nematode Bioassay

This exemplified method employs binary vectors containing fragments of the 50657480 target gene. The vector consists of an antisense fragment of the target 50657480 gene, a spacer, a sense fragment of the target gene and a vector backbone. The sequence of the 50657480 cDNA clone is described as SEQ ID NO:1. The target gene fragment described by SEQ ID NO:2 corresponding to nucleotides 7 to 483 of SEQ ID NO:1 was used to construct the binary vector RAW464. In RAW464 the dsRNA for the 50657480 target gene was expressed under a syncytia or root preferred promoter p-At5g05340 (US-provisional application No. 60/899,693 SEQ ID NO: 6), a peroxidase gene promoter. This promoter drives transgene expression preferentially in roots and/or syncytia or giant cells. The plant selectable marker in the binary vectors is a herbicide-resistant form of the acetohydroxy acid synthase (AHAS) gene from Arabidopsis thaliana driven by the native Arabidopsis AHAS promoter (Sathasivan et al., Plant Phys. 97:1044-50, 1991). ARSENAL (imazapyr, BASF Corp, Florham Park, N.J.) was used as the selection agent.

The binary vector RAW464 was transformed into Agrobacterium rhizogenes K599 strain by electroporation and transgenic hairy roots were generated using known methods. Several independent transgenic hairy root lines were generated from transformation. Non-transgenic hairy roots from soybean cultivar Williams 82 (SCN susceptible) and Jack (SCN resistant) were also generated by using non-transformed A. rhizogenes, to serve as controls for nematode growth in the assay. Hairy root cultures of each line were inoculated with SCN race 3 second stage juveniles (J2). Four weeks after nematode inoculation, the cyst number in each well was counted. For RAW464 transgenic root lines there were multiple lines demonstrating mean cyst counts around 6-7 and 11-18 as compared to a mean cyst count of 24 and 26 for the susceptible line Williams 82 (W82) and 1 and 1 for the known resistant line, Jack, respectively. These bioassay results indicate that the double stranded RNA expressed in RAW464 results in reduced cyst count.

### Example 3

### RACE to Determine Full Transcribed Sequence for 50657480 (SEQ ID NO:1)

Amplification of full-length transcript sequence corresponding to the cDNA sequence described by 50657480 (SEQ ID NO:1) was achieved using the GeneRacer Kit (L1502-01) from Invitrogen by following the manufacturers instructions. The primers used for the primary PCR reaction are described by SEQ ID NOs 12 and 14. The secondary nested PCR reaction primers are described by SEQ ID NOs 13 and 15.

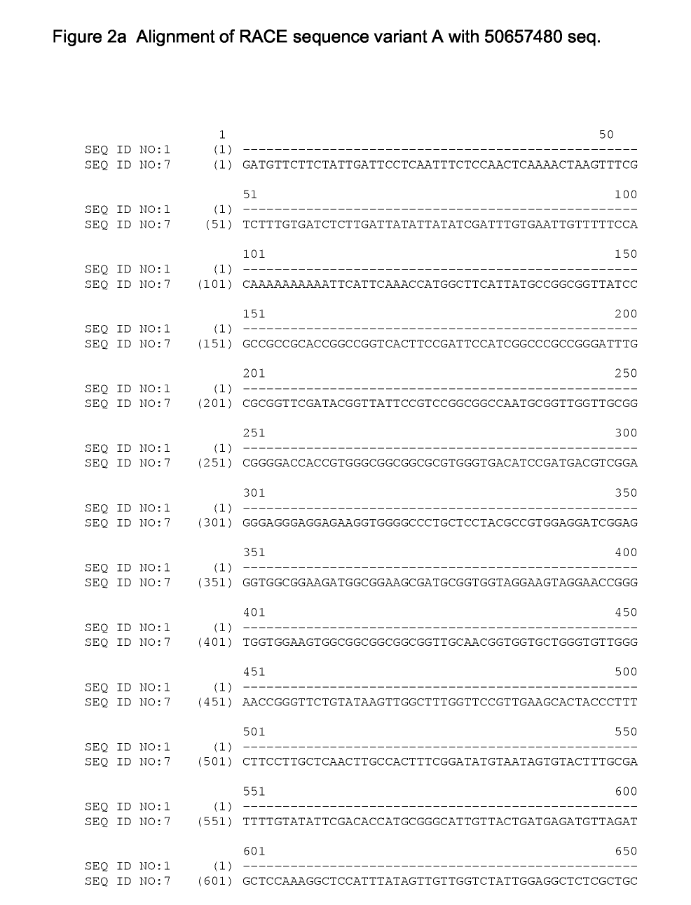

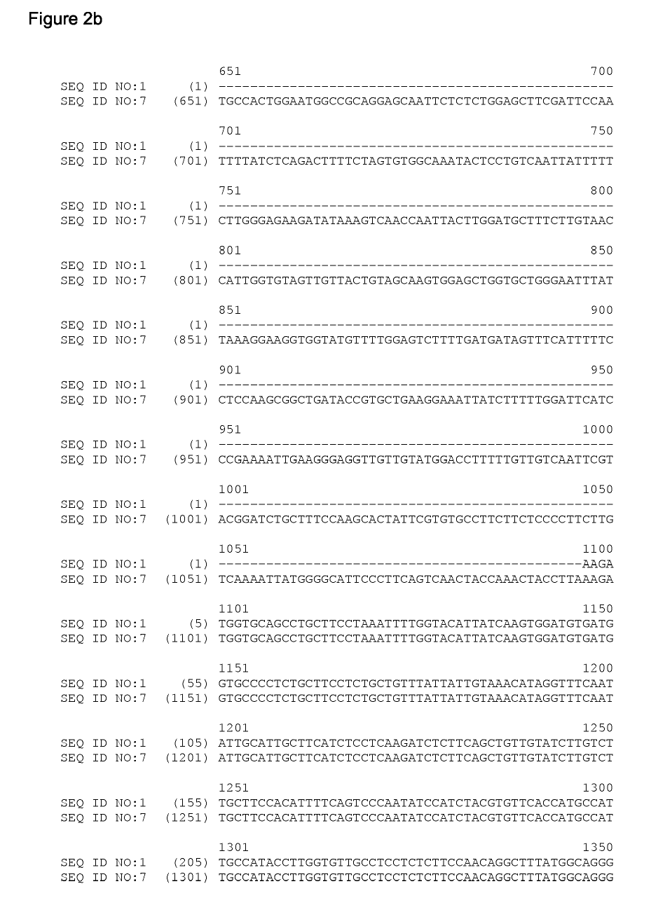

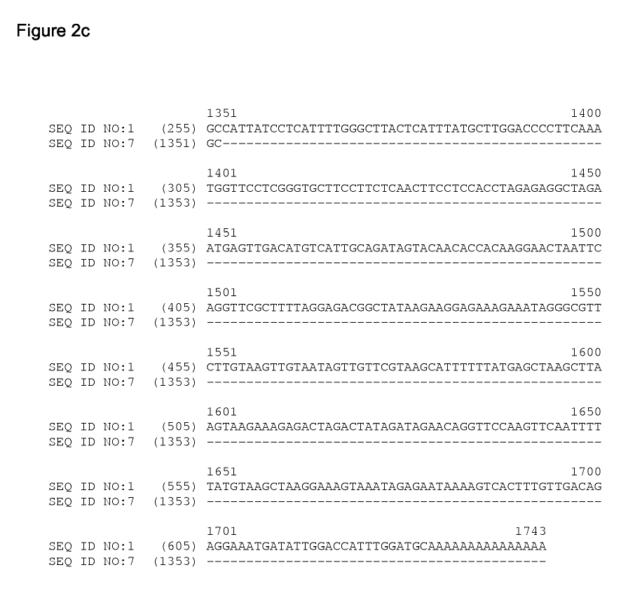

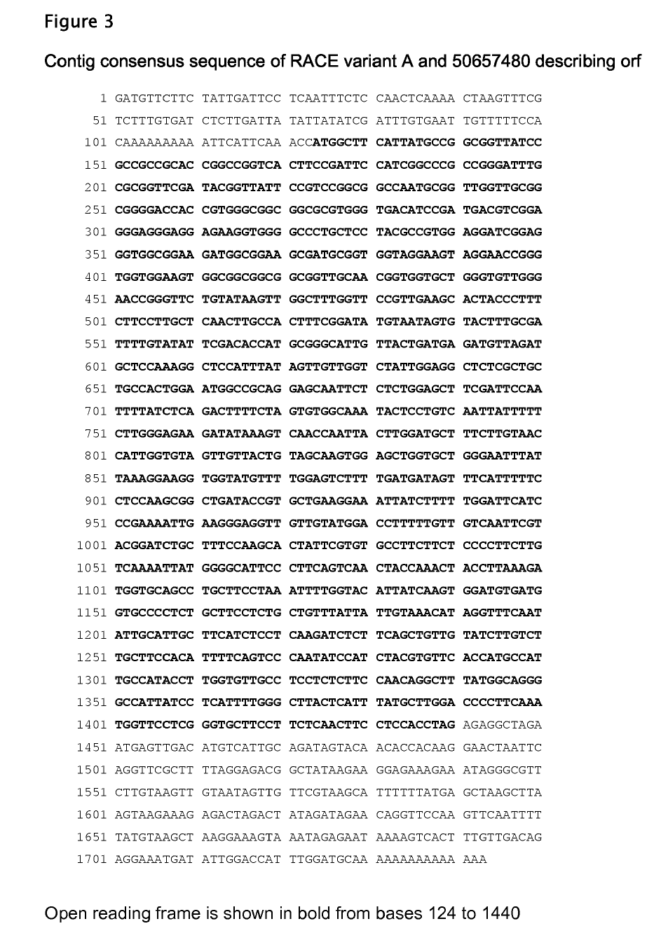

As shown in FIG. 2, SEQ ID NO:7 is the 5′ fragment of 50657480. Based on the alignment of SEQ ID NO:7 and SEQ ID NO:1 shown in FIG. 2, a putative full length contig sequence was isolated and is described by SEQ ID NO:8. There is an open reading frame in SEQ ID NO:8 contig sequence that spans from bases 124 to 1440 as shown in FIG. 3. The open reading frame sequence is described by SEQ ID NO:9. The amino acid sequence of the open reading frame described by SEQ ID NO:9 is shown as SEQ ID NO:10.

### Example 4

### Description of Homologs (Nucleotide and AA)

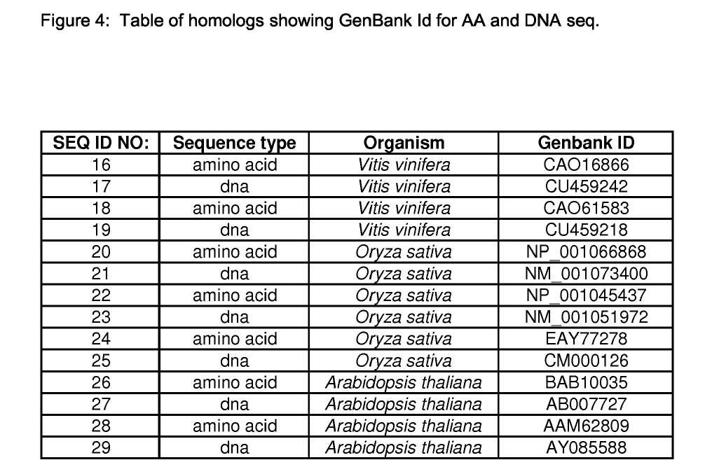

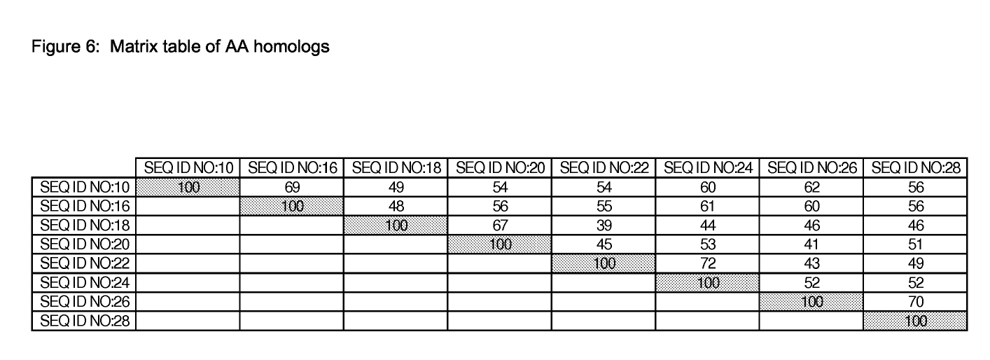

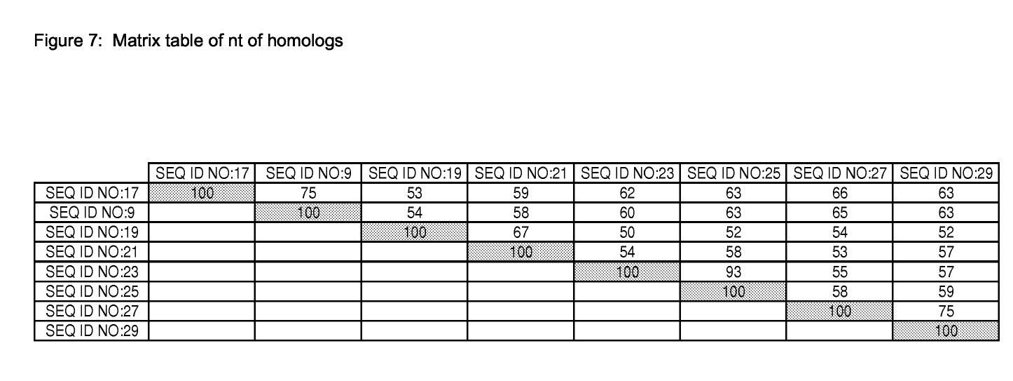

As disclosed in Example 3, the putative full length transcript sequence of the gene corresponding to SEQ ID NO:1 contains an open reading frame with the amino acid sequence disclosed as SEQ ID NO:10. The identification of gene homologs to the amino acid sequence described by SEQ ID NO:10 identifies additional sequences. A sample of genes with amino acid and DNA sequences homologous to SEQ ID NO:10 and SEQ ID NO:9, respectively, were identified and are described by SEQ ID NOs 16 to 29 and shown in FIG. 4. The amino acid alignment of the identified truncated homologs to SEQ ID NO:10 is shown in FIG. 5. A matrix table showing the amino acid percent identity of the identified homologs and SEQ ID NO:10 to each other is shown in FIG. 6. A matrix table showing the DNA sequence percent identity of the identified homologs and SEQ ID NO:9 to each other is shown in FIG. 7.

Those skilled in the art will recognize, or will be able to ascertain using no more than routine experimentation, many equivalents to the specific embodiments of the invention described herein. Such equivalents are intended to be encompassed by the following claims.

What is claimed is:

1. A dsRNA molecule comprising a) a first strand comprising a sequence substantially identical to a portion of a a 50657480 gene, a 50657480-like gene or a 50657480-homolog and b) a second strand comprising a sequence substantially complementary to the first strand.

2. The dsRNA molecule of claim 1, wherein the portion of the 50657480 gene, 50657480-like gene or a 50657480-homolog is a sequence selected from the group consisting of:
a) a polynucleotide comprising a sequence as set forth in SEQ ID NO:1, nucleotides 7 to 483 of SEQ ID NO: 1, SEQ ID NO: 7, nucleotides 1 to 1096 of SEQ ID NO:7 or SEQ ID NO:8;
b) a polynucleotide comprising a sequence having at least 80% sequence identity to SEQ ID NO.1, nucleotides 7 to 483 of SEQ ID NO: 1, SEQ ID NO: 7, nucleotides 1 to 1096 of SEQ ID NO:7 or SEQ ID NO:8;
c) a polynucleotide that hybridizes under stringent conditions to a polynucleotide comprising a sequence as set forth in SEQ ID NO:1 nucleotides 7 to 483 of SEQ ID NO: 1, SEQ ID NO: 7, nucleotides 1 to 1096 of SEQ ID NO:7 or SEQ ID NO:8,
d) a polynucleotide being obtainable with primers having the sequence as set forth in SEQ ID NO: 4, 5, 14, or 15,
e) a polynucleotide comprising a sequence having at least 50% sequence identity to a polynucleotide coding for a nucleotide sequence as set forth in SEQ ID NO: 9, 17, 19, 21, 23, 25, 27 or 29.
f) a polynucleotide comprising a sequence having at least 40% sequence identity to a polynucleotide coding for an amino acid sequence as set forth in SEQ ID NO:10, 16, 18, 20, 22, 24, 26 or 28.

3. The dsRNA molecule of claim 1, wherein the portion of the target gene is from about 19 to 500 nucleotides.

4. A pool of dsRNA molecules comprising a multiplicity of RNA molecules each comprising a double stranded region having a length of about 19 to 24 nucleotides, wherein said RNA molecules are derived from a polynucleotide being substantially identical to a portion of a 50657480 gene, a 50657480-like gene or a 50657480-homolog.

5. A pool of dsRNA molecules as claimed in claim 4, wherein said RNA molecules are derived from a polynucleotide selected from the group consisting of: a) a polynucleotide comprising a sequence as set forth in SEQ ID NO:1, nucleotides 7 to 483 of SEQ ID NO: 1, SEQ ID NO: 7, nucleotides 1 to 1096 of SEQ ID NO:7 or SEQ ID NO:8;
b) a polynucleotide comprising a sequence having at least 80% sequence identity to SEQ ID NO.1, nucleotides 7 to 483 of SEQ ID NO: 1, SEQ ID NO: 7, nucleotides 1 to 1096 of SEQ ID NO:7 or SEQ ID NO:8;
c) a polynucleotide that hybridizes under stringent conditions to a polynucleotide comprising a sequence as set forth in SEQ ID NO:1, nucleotides 7 to 483 of SEQ ID NO: 1, SEQ ID NO: 7, nucleotides 1 to 1096 of SEQ ID NO:7 or SEQ ID NO:8
d) a polynucleotide being obtainable with primers having the sequence as set forth in SEQ ID NO: 4, 5, 14, or 15,
e) a polynucleotide comprising a sequence having at least 50% sequence identity to a polynucleotide coding for a nucleotide sequence as set forth in SEQ ID NO: 9, 17, 19, 21, 23, 25, 27 or 29.
f) a polynucleotide comprising a sequence having at least 40% sequence identity to a polynucleotide coding for an amino acid sequence as set forth in SEQ ID NO:10, 16, 18, 20, 22, 24, 26 or 28.

6. A transgenic plant capable of expressing a dsRNA that is substantially identical to a portion of a 50657480-like gene or a 50657480-homolog.

7. The transgenic plant of claim 6, wherein the 50657480 gene, 50657480-like gene or 50657480-homolog comprises a sequence selected from the group consisting of:
a) a polynucleotide comprising a sequence as set forth in SEQ ID NO:1, nucleotides 7 to 483 of SEQ ID NO: 1, SEQ ID NO: 7, nucleotides 1 to 1096 of SEQ ID NO:7 or SEQ ID NO:8;
b) a polynucleotide comprising a sequence having at least 80% sequence identity to SEQ ID NO.1, nucleotides 7 to 483 of SEQ ID NO: 1, SEQ ID NO: 7, nucleotides 1 to 1096 of SEQ ID NO:7 or SEQ ID NO:8;
c) a polynucleotide that hybridizes under stringent conditions to a polynucleotide comprising a sequence as set forth in SEQ ID NO:1, nucleotides 7 to 483 of SEQ ID NO: 1, SEQ ID NO: 7, nucleotides 1 to 1096 of SEQ ID NO:7 or SEQ ID NO:8,
d) a polynucleotide being obtainable with primers having the sequence as set forth in SEQ ID NO: 4, 5, 14, or 15,
e) a polynucleotide comprising a sequence having at least 50% sequence identity to a polynucleotide coding for a nucleotide sequence as set forth in SEQ ID NO: 9, 17, 19, 21, 23, 25, 27 or 29.
f) a polynucleotide comprising a sequence having at least 40% sequence identity to a polynucleotide coding for an amino acid sequence as set forth in SEQ ID NO:10, 16, 18, 20, 22, 24, 26 or 28.

8. A transgenic plant capable of expressing a pool of dsRNA molecules, wherein pool of RNA molecules each comprising a double stranded region having a length of about 19-24 nucleotides, wherein the RNA molecules are derived from a polynucleotide substantially identical to a portion of a 50657480 gene, a 50657480-like gene or a 50657480-homolog.

9. The transgenic plant of claim 8, wherein said RNA molecules are derived from a polynucleotide selected from the group consisting of:
a) a polynucleotide comprising a sequence as set forth in SEQ ID NO:1, nucleotides 7 to 483 of SEQ ID NO: 1, SEQ ID NO: 7, nucleotides 1 to 1096 of SEQ ID NO:7 or SEQ ID NO:8;
b) a polynucleotide comprising a sequence having at least 80% sequence identity to SEQ ID NO.1, nucleotides 7 to 483 of SEQ ID NO: 1, SEQ ID NO: 7, nucleotides 1 to 1096 of SEQ ID NO:7 or SEQ ID NO:8;
c) a polynucleotide that hybridizes under stringent conditions to a polynucleotide comprising a sequence as set forth in SEQ ID NO:1, nucleotides 7 to 483 of SEQ ID NO: 1, SEQ ID NO: 7, nucleotides 1 to 1096 of SEQ ID NO:7 or SEQ ID NO:8
d) a polynucleotide being obtainable with primers having the sequence as set forth in SEQ ID NO: 4, 5, 14, or 15,
e) a polynucleotide comprising a sequence having at least 50% sequence identity to a polynucleotide coding for a nucleotide sequence as set forth in SEQ ID NO: 9, 17, 19, 21, 23, 25, 27 or 29.
f) a polynucleotide comprising a sequence having at least 40% sequence identity to a polynucleotide coding for an amino acid sequence as set forth in SEQ ID NO:10, 16, 18, 20, 22, 24, 26 or 28.

10. A method of making a transgenic plant capable of expressing a pool of dsRNA molecules that is substantially identical to a portion of a 50657480 gene, a 50657480-like gene or a 50657480-homolog in a plant, said method comprising the steps of:
a) preparing a nucleic acid sequence having a region that is substantially identical to a portion of a 50657480 gene, a 50657480-like gene or a 50657480-homolog, wherein the nucleic acid is able to form a double-stranded transcript of a portion of a 50657480-like gene or a 50657480-homolog once expressed in the plant;
b) transforming a recipient plant with said nucleic acid;
c) producing one or more transgenic offspring of said recipient plant; and
d) selecting the offspring for expression of said transcript.

11. The method of claim 10, wherein the target gene comprises a sequence selected from the group consisting of:
a) a polynucleotide comprising a sequence as set forth in SEQ ID NO:1, nucleotides 7 to 483 of SEQ ID NO: 1, SEQ ID NO: 7, nucleotides 1 to 1096 of SEQ ID NO:7 or SEQ ID NO:8;
b) a polynucleotide comprising a sequence having at least 80% sequence identity to SEQ ID NO.1, nucleotides 7 to 483 of SEQ ID NO: 1, SEQ ID NO: 7, nucleotides 1 to 1096 of SEQ ID NO:7 or SEQ ID NO:8;
c) a polynucleotide that hybridizes under stringent conditions to a polynucleotide comprising a sequence as set forth in SEQ ID NO:1, nucleotides 7 to 483 of SEQ ID NO: 1, SEQ ID NO: 7, nucleotides 1 to 1096 of SEQ ID NO:7 or SEQ ID NO:8
d) a polynucleotide being obtainable with primers having the sequence as set forth in SEQ ID NO: 4, 5, 14, or 15,
e) a polynucleotide comprising a sequence having at least 50% sequence identity to a polynucleotide coding for a nucleotide sequence as set forth in SEQ ID NO: 9, 17, 19, 21, 23, 25, 27 or 29.
f) a polynucleotide comprising a sequence having at least 40% sequence identity to a polynucleotide coding for an amino acid sequence as set forth in SEQ ID NO:10, 16, 18, 20, 22, 24, 26 or 28.

12. The method of claim 10, wherein the portion of the 50657480 gene, 50657480-like gene or 50657480-homolog e is from about 19 to about 500 nucleotides.

13. The method of claim 10, wherein the plant is selected from the group consisting of: soybean, potato, tomato, peanuts, cotton, cassava, coffee, coconut, pineapple, citrus trees, banana, corn, rape, beet, sunflower, sorghum, wheat, oats, rye, barley, rice, green bean, lima bean, pea, and tobacco.

14. The method of claim 10 wherein the plant is a soybean plant.

15. A method of conferring nematode resistance to a plant, said method comprising the steps of:
a) preparing a nucleic acid sequence having a region that is substantially identical to a portion of a 50657480 gene, a 50657480-like gene or a 50657480-homolog, wherein the nucleic acid is able to form a double-stranded transcript of a portion of a 50657480-like gene or a 50657480-homolog once expressed in the plant;
b) transforming a recipient plant with said nucleic acid;
c) producing one or more transgenic offspring of said recipient plant; and
d) selecting the offspring for nematode resistance.

16. An expression vector comprising a sequence substantially identical to a portion of a 50657480 gene, a 50657480-like gene or a 50657480-homolog.

17. An expression as claimed in claim 16, comprising a second sequence substantially complementary to the first strand, capable of forming a dsRNA, when both sequences are transcribed.

18. An expression as claimed in claim 16, comprising a root-preferable promoter.
